(19) United States
(12) Patent Application Publication
(10) Pub. No.: US 2020/0363433 A1
(43) Pub. Date: Nov. 19, 2020

(54) METHODS OF DIAGNOSING AND TREATING PARTICULAR CAUSAL COMPONENTS OF CHRONIC PAIN IN A PATIENT

(71) Applicant: Ethos Research & Development, LLC, Newport, KY (US)

(72) Inventors: Joshua Adam Gunn, Covington, KY (US); Brian Kincaid, Cincinnati, OH (US)

(21) Appl. No.: 16/834,435

(22) Filed: Mar. 30, 2020

Related U.S. Application Data
(60) Provisional application No. 62/923,032, filed on Oct. 18, 2019.
(60) Provisional application No. 62/825,932, filed on Mar. 29, 2019.

(51) Int. Cl. G01N 33/70

(52) U.S. Cl. CPC G01N 33/70; G01N 2800/7009; G01N 2800/2842; G01N 2800/7095; G01N 2800/60

(57) ABSTRACT

The present disclosure teaches systems and methods of diagnosing and treating the distinct biologic components that contribute to chronic pain symptoms experienced by patients. A biologic sample is obtained from a patient. Levels of two or more biomarkers (e.g., methylmalonic acid, homocysteine, xanthurenic acid, 3-hydroxypropyl mercapturic acid (3-HPMA), pyroglutamate, hydroxymethylglutarate (HMG), quinolinic acid, kynurenine acid, 5-hydroxyindoleacetate (5-HIAA), vanilmandelate (VMA), and ethylmalonic acid) in the biologic sample are experimentally determined. Based on the existence of abnormal results in one or more biomarkers the patient is diagnosed as having the nerve health, oxidative stress, chronic inflammation pain, and/or neurotransmitter pain components to their chronic pain. Based on the resulting diagnoses administration of certain support compounds is directed. The patient may retest after a sufficient period of time to observe any longitudinal differences in the test results and adjust treatment accordingly. Further, the biomarker data gathered from pain-neutral and chronic pain patients (particularly those using opioid therapies) will be used to characterize biochemistries going forward.

### CROSS-REFERENCE TO RELATED APPLICATIONS

This application claims priority to U.S. Provisional Application Ser. No. 62/825,932 filed on Mar. 29, 2019, and to U.S. Provisional Application Ser. No. 62/923,032 filed on Oct. 18, 2019, the entire contents of which are hereby expressly incorporated herein by reference.

### TECHNICAL FIELD

The present disclosed relates generally to the field of medical diagnostics and treatment and more particularly to methods, apparatuses, and systems of quantifying biochemical abnormalities in a patient presenting with chronic pain to diagnose and effectively treat each particular causal component of that patient's chronic pain.

### BACKGROUND

Chronic pain is a form of pain that persists past normal healing time and hence lacks the acute warning function of physiological nociception. Pain that persists for or recurs for more than 3 to 6 months is typically considered chronic in nature. We use this definition for “chronic pain” in the present application.

Chronic pain is estimated to affect 20% of people worldwide and account for 15-20% of all physician visits. According to data published from the Center for Disease Control (CDC) in 2016, approximately 20.4% of the US population (i.e., 50 million people) experiences chronic pain with 8% (i.e., nearly 20 million) experiencing “High Impact Chronic Pain.” Chronic pain has been linked to restricted mobility, opioid dependency, anxiety, depression and reduced quality of life.

Recent reviews indicate that total societal costs of chronic pain in the United States range from $560 to $635 billion annually. In other words, the cost of chronic pain due to direct medical treatments and lost productivity represents a greater economic burden than many of the nation's priority health conditions such as heart disease, cancer and diabetes. Despite the soaring costs of treating chronic pain, complete relief is uncommon due to the limited efficacy of current treatments.

Moreover, treatment of pain, especially chronic pain is further complicated by the lack of a reliable quantitative measures of pain. It is generally understood that the biologic and physiologic mechanisms involved in chronic pain are different in each patient and may change over time. The complexity of these mechanisms make may further complicate testing associated with chronic pain, particularly to arrive at a test or series of test that can accurately and consistently assess and even quantify the nature of the chronic pain in each patient over time.

For instance, while it is widely recognized that acute pain acutely increases blood pressure, other factors also cause acute increases in blood pressure (e.g., diet, dehydration, medications, thyroid problems, sleep apnea, stress). Even if the contribution of acute pain to increases in blood pressure could be isolated (which it presently cannot), the effect of chronic pain on blood pressure is not well understood. Some researchers even believe that there may be an inverse relationship between resting blood pressure levels and pain sensitivity. Further, even if blood pressure changes could be correlated to changes in chronic pain, this measure would still fail to provide insight into the biomechanism contributing to the pain.

To date, subjective pain ratings have played a key role in the medical diagnosis and treatment of chronic pain. However, profound individual differences in sensitivity make subjective pain ratings unreliable and generally complicate the medical diagnosis and, thus, any resulting treatment. Due to the subjective nature of current pain assessments, limited efficacy of treatment options and risks associated with opioid abuse and diversion, a need exists for an objective pain measurement to assist with chronic pain management.

Moreover, patients experiencing chronic pain may have difficulties recognizing and/or expressing changes in their chronic pain. This may be due to the patient's age, health status (e.g., mute, aphasia, dementia), or even just an inability to successfully articulate to others information regarding their pain. Many patients with chronic pain are treated with opioids. The ability to objectively assess and observe changes in these patient's chronic pain may help avoid the various problems associated with opioid use. For instance, a patient's increased chronic pain may be due to a separate mechanism than the pathology that led to the prescription of opioids in the first instance. In such an example, process and system that separately assesses disparate discrete pain mechanisms in the body could help avoid the unnecessary opioid dosage increases and/or unnecessary prolonged use of opioids. Thus, it would be beneficial to develop a diagnostic tool that can quantitatively assess changes associated with chronic pain over time. And it would be further beneficial if that diagnostic tool could quantitatively assess disparate discrete pain mechanisms separately.

Although there have been substantial advances in our understanding of chronic pain pathophysiology in recent decades, most of the research has been focused on identifying relevant biochemical pathways and/or biomarkers toward developing novel pain drugs. While these research efforts are likely to prove beneficial to the pain community in the years to come, there is an immediate need for improved pain management. There is an associated need to objectively identify underlying causes of chronic pain toward classifying patient: by the cause(s) of their chronic pain and further need to identify new modes of pain treatment that address each of these variety of causes.

Given the biopsychosocial complexity of chronic pain and the frequency of comorbid diagnoses related to depression and anxiety, it is not surprising that biomarker discovery related to physical pain has lagged behind other specialties in recent decades. Some researchers have even contested the validity of the search and concluded that finding biomarkers for pain is a sheer impossibility as pain, by definition, is a subjective experience. In fact, most, if not all clinical researchers would agree that the experience of pain is always subjective and will never be quantifiable. Successful identification of mechanistic biomarkers of pain (markers that reveal which pathophysiological mechanism is responsible for the pain) would not only improve our understanding and ability to accurately diagnose pain disorders, but could also pave the way for the development of disease-modifying pain drugs.

While the endeavor to discover clinically suitable biomarkers of pain is no doubt a challenging journey, the chronic pain experience will always be, in large part, subjective, and no biomarker will ever replace patient self-reporting. Rather, mechanistic biomarkers of pain could provide physicians with objective information pertaining to the biochemical pathway responsible for the discomfort. Such information could allow providers to move away from the trial-and-error symptom control model and allow for recommending medications and interventional procedures that modulate the course of disease.

In the end, the medical community's ability to study pain, and especially chronic pain, has been impeded by the lack of objective, quantitative measures that could further science's understanding of the relationship between various chronic pain mechanisms and the body. A process and/or system that provides normalized quantitative longitudinal data that scientists could use to analyze the relationship between various biologic processes and chronic pain would be extremely useful in quickly advancing medical knowledge and treatment.

The present disclosure seeks to address one or more of the aforementioned needs in the art.

### BRIEF SUMMARY OF THE INVENTION

Disclosed are methods, apparatuses, and systems for detecting abnormal biochemistry indicative of a plurality of the contributing causes of chronic pain. The disclosed methods, apparatuses, and systems place quantitative values on each abnormal biochemistry and thus identify a particular state of that patient's chronic pain, which allows for informed selection and initiation of a therapeutic regimen in order to reduce the patient's chronic pain. The disclosed methods, apparatuses, and systems further allow for the longitudinal comparison of these quantitative results such that the therapeutic regimen can be properly adjusted in view of material changes in the results.

A composite biomarker score may be generated for a patient having chronic pain, which indicates the severity of atypical, pro-pain biochemistry. This composite biomarker score may also be associated with sub-scores for each of a plurality of mechanisms that are believed may contribute to chronic pain (e.g., nerve health, oxidative stress, chronic inflammation, neurotransmitter status). Thus, the composite biomarker score provides a context by which the patient and medical professional can understand the patient's relative level of chronic pain. The composite biomarker (and particularly is constituent components) can be used to guide medical professionals in determining treatment. Moreover, by assessing changes in a patient's composite biomarker score over time (and particularly following treatment for at least one or more of the observed chronic pain mechanisms), the medical professional would be able to better manage that a patient's chronic pain. The composite biomarker score provides a quantitative value to more accurately assess and document the level of chronic pain in a patient. The composite biomarker score also helps medical professionals assess differences between patients, treatments, and even medical practices.

The methods, apparatuses, and systems may facilitate ascertaining the role of abnormal or atypical biochemistry as a cause of pain in an individual patient by producing a composite score using thresholds and data obtained from pain-positive cohorts and pain-negative cohorts. The data obtained by these novel processes, apparatuses, and systems from across an entire patient population (including pain-positive and pain-negative cohorts) can provide a general chronic pain model that should be extremely valuable to public health professionals toward better managing the chronic pain crisis. In turn, it is the hope that the resulting generalized chronic pain management models will help stem the tide of the opioid crisis, as physician are provided with specific quantitative data to more precisely manage the plurality of mechanisms that contribute to chronic pain.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A, 1B, 1C, 1D, 1E, 1F, 1G, 1H, 1I, 1J and 1K collectively illustrate various potential aspects of a method of diagnosing and treating distinct biologic components contributing to chronic pain experienced by a patient.

FIG. 2 is a diagrammatic view of the hardware for an exemplary user device and for an exemplary server device (co-located with laboratory equipment) wherein the exemplary user device and exemplary server device are operably connected to one another via a network (such as the Internet, a WAN, or a LAN) for use in the method of diagnosing and treating distinct biologic components contributing to chronic pain experienced by a patient.

FIGS. 3A, 3B, 3C, 3D, 3E, and 3F illustrate various aspects of an exemplary graphical user interface associated with the output device of the exemplary user device as well as the output generated for a printer, such as the illustrated laser printer, for a particular patient who exhibited a low composite pain index.

FIGS. 4A, 4B, 4C, and 4D, illustrate various aspects of an exemplary graphical user interface associated with the output device of the exemplary user device for a particular patient who exhibited a high composite pain index.

FIG. 5 is an illustration of novel individually sealed, frangible packaging for a plurality nerve health support compounds.

FIG. 6 is an illustration of novel individually sealed, frangible packaging for a plurality chronic inflammation support compounds.

FIG. 7 is an illustration of novel individually sealed, frangible packaging for a plurality oxidative stress support compounds.

FIG. 8 is a partial cutaway illustration of one potential exemplary embodiment of a patient sample kit that includes the various biomechanism support compounds

FIG. 9 illustrates the ROC curve of FPI score severity and clinical assessments of chronic pain (i.e., SF-36 scores) observed from the results of a clinical study.

FIG. 10 illustrates the comparison of means (non-parametric t-test) of calculated FPI score observed from the results of a clinical study.

FIG. 11 illustrates the calculated FPI score (healthy vs. pain) observed from the results of a clinical study.

FIG. 12 illustrates the comparison of means (non-parametric t-test) of calculated FPI scores and clinical assessments of chronic pain between pain patients with high FPI and low FPI severity scores observed from the results of a clinical study.

FIG. 13 illustrates the association between SF-36 scores and FPI severity among chronic pain patients observed from the results of a clinical study.

### DETAILED DESCRIPTION

Before explaining at least one embodiment of the presently disclosed and claimed inventive concepts in detail, it is to be understood that the presently disclosed and claimed inventive concepts are not limited in their application to the details of construction, experiments, exemplary data, and/or the arrangement of the components set forth in the following description or illustrated in the drawings. The presently disclosed and claimed inventive concepts are capable of other embodiments or of being practiced or carried out in various ways. Also, it is to be understood that the phraseology and terminology employed herein is for purpose of description and should not be regarded as limiting.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

The use of the terms “at least one” and “one or more” will be understood to include one as well as any quantity more than one, including but not limited to each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “analyte” in the context of the present teachings can mean any substance to be measured, and can encompass organic acids, amino acids, neurotransmitter metabolites, neurotransmitter precursors, biomarkers, markers, nucleic acids, electrolytes, metabolites, proteins, sugars, carbohydrates, fats, lipids, cytokines, chemokines, growth factors, proteins, peptides, nucleic acids, oligonucleotides, metabolites, mutations, variants, polymorphisms, modifications, fragments, subunits, degradation products and other elements.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

“Biomarker,” as used herein, is a characteristic that can be objectively measured and evaluated as an indicator of normal biological processes, pathogenic processes, or pharmacologic response to a therapeutic intervention. Biomarkers may comprise small compound “metabolites” which may be measured in biological fluids including, but not limited to whole blood, serum, plasma, urine, oral fluid, or sweat. Biomarkers are employed across most medical specialties for various purposes including, but not limited to: identifying patients at risk of developing disease; disease diagnosis; prognosis; evaluation of treatment response and early stage drug development.

As used herein, the phrase “biomarker profile” means the combination of a patient's biomarker levels found in one or more of their biological specimens or samples.

In one aspect, the term “atypical” refers to quantitative levels of metabolomics biomarkers that are outside the 95% double sided reference or normal ranges that have been established from pain-negative subjects.

As used herein, the term “higher likelihood,” as used, for example, in connection with a significantly “higher likelihood,” means that there is a higher likelihood that atypical biochemistry, or abnormal biomarker findings, are a causative or contributing factor in that patient's pain compared to a pain-negative individual. The higher likelihood may be relative or absolute and may be expressed qualitatively or quantitatively. For example, a higher likelihood may be expressed as simply determining the patient's biomarker levels and placing the patient in a “higher likelihood” category, based upon previous population studies. Alternatively, a numerical expression of the subject's increased risk may be determined based upon the metabolite profile. As used herein, examples of expressions of a higher likelihood include, but are not limited to, odds, probability, odds ratio, p-values, attributable likelihood, composite biomarker score or index, relative frequency, positive predictive value, negative predictive value and relative likelihood.

“Measure,” “measuring” or “measurement” in the context of the present teachings refers to determining the presence, absence, quantity, amount, or effective amount of a substance in a clinical or subject-derived sample, including the concentration levels of such substances, or evaluating the values or categorization of a subject's clinical parameters.

A “score” is a value or set of values selected so as to provide a quantitative measure of a variable or variables characteristic of a patient's condition, and/or to discriminate, differentiate or otherwise characterize a patient's condition. The value(s) comprising the score can be based on, for example, a measured amount of one or more biomarkers in a biologic sample obtained from a subject. The value(s) comprising the score may also be based on clinical parameters or assessments. In some instances, the value(s) comprising the scope may any combination of the foregoing.

Disclosed are methods and systems for diagnosing and treating distinct biologic components contributing to chronic pain experienced by a patient. Also disclosed are ancillary methods of combining quantitative biomarker levels into a composite biomarker score. The methods may comprise obtaining at least one biological sample from a patient with chronic pain to determine the level of two or more selected biomarkers and then diagnosing the existence of abnormal biomarkers indicative of the presence of a particular chronic pain mechanism (e.g. nerve health, oxidative stress, chronic inflammation, and neurotransmitter status). The method may also comprise normalizing the biological sample based on its creatinine values and then combining the normalized levels into a composite value/score of the detected biomarkers. The various methods may further comprise comparing the value of a patient's composite score with a composite score obtained from pain-negative subjects (patients that do not have chronic pain).

In one aspect, the amount of the one or more biomarker(s) disclosed herein can be measured in a sample and used to derive a score, which score may then then compared to a “normal” or “control” level or value, utilizing techniques such as, e.g., reference or discrimination limits or risk defining thresholds, in order to define cut-off points and/or abnormal values for inflammatory disease. The normal level then is the level of one or more biomarkers or combined biomarker indices typically found in a subject who is not suffering from chronic pain. Other terms for “normal” or “control” are, e.g., “reference,” “index,” “baseline,” “standard,” “healthy,” “pre-disease,” “pain-negative” etc. Such normal levels can vary, based on whether a biomarker is used alone or in a formula combined with other biomarkers to output a score. Alternatively, the normal level can be a database of biomarker patterns from previously tested subjects prior to reporting or being diagnosed with chronic pain. Reference (normal, control) values can also be derived from, e.g., a control subject or population whose pain status is known.

In certain aspects, the patient for which the composite score is determined may be one who is currently being treated with an opioid analgesic. A composite biomarker score significantly above the threshold or thresholds established from a pain-negative cohort indicates that there is a likelihood that abnormal biochemistry may be a cause or contributing factor in that patient's painful symptoms, and that correction or partial correction of the aberrant biochemistry may be used in lieu of or in addition to opioid treatment. In other word, the methods disclosed herein may be used to identify chronic pain patients for which continued opioid treatment can be reduced or altogether avoided by correction of an underlying defect in the individual patient's biochemistry.

In one aspect, the disclosed methods may be used to determine a possible origin or cause of pain in a patient with chronic pain in the absence of an identified pathological causation or etiology.

In another aspect, the disclosed methods may be employed to evaluate underlying biochemistry in a patient with chronic pain syndromes including, but not limited to neuropathic pain, nociceptive pain, complex regional pain syndrome, fibromyalgia, central sensitization syndrome, sensory hypersensitivity, chronic inflammatory pain, chronic visceral pain, chronic somatic pain and chronic refractory pain with unknown etiology.

Objective biomarkers are the core elements of personalized medicine and the identification and validation of markers to assist with the diagnosis and treatment of chronic pain would significantly reduce healthcare costs worldwide.

Biomarkers are commonly employed in medicine to diagnose or determine the presence or absence of a particular disease. If a biomarker is determined to possess statistically significant discriminative power to distinguish between disease-positive cohorts and disease-negative cohorts it may be employed as a single diagnostic biomarker for that disease. In such cases, a patient may be diagnosed with a disease if their biomarker level is 2-3 standard deviations from the mean value obtained from the disease-negative cohort. Oftentimes, however, individual biomarkers do not possess sufficient discriminatory power to determine the presence or absence of a disease as a single measurement. Rather, a panel or plurality of biomarkers may be employed to generate a biomarker ‘signature’ which may better describe the health status of a patient.

The quantity of one or more biomarkers of the present teachings can be indicated as a value. The value can be one or more numerical values resulting from the evaluation of a sample, and can be derived, e.g., by measuring level(s) of the biomarker(s) in a sample by an assay performed in a laboratory, or from dataset obtained from a provider such as a laboratory, or from a dataset stored on a server. Biomarker levels can be measured using any of several techniques known in the art. The present teachings encompass such techniques. Techniques to quantitatively measure levels of metabolomics biomarkers described herein are well known to the skilled technician, and exemplary methods are not intended to limit the scope of the invention. In one aspect, levels of the individual metabolomics biomarkers may be assessed using mass spectrometry coupled with high-performance liquid chromatography (HPLC), ultra-performance liquid chromatography (UPLC). Other methods of quantitatively determining metabolomics biomarker levels include biological methods such as ELISA, EIA, RIA assays.

Quantitative levels of individual metabolomics biomarkers in the biomarker profile may be expressed as absolute or relative values and may or may not be expressed in relation to an internal standard or another compound known to be present at a constant concentration in the biological sample. If quantitative levels are determined using a standard additions or internal standard, the standard may be added to the test sample prior to, during or after sample processing.

Quantitative levels of metabolomics biomarkers determined in urine samples may be normalized to determine a relative level. For example, the methods herein include normalization to creatine, and are referred to as ‘creatinine normalized’ values to account for fluctuations in urinary output and urine concentration.

The search for pain biomarkers at the heart of the present disclosure has been focused on identifying objective, measurable correlates to the neurobiological processes underlying painful conditions with the aim of enabling chronic pain diagnoses and treatments to be based on underlying pathophysiological mechanisms rather than symptomology. While the successful identification of any pain specific biomarker signifies an advancement in our understanding of pain pathophysiology, the most important and impactful biomarkers are likely to be those that can be modulated to change the course of disease.

Individual components of an exemplary metabolomics biomarker profile for chronic pain include, but are not limited to: (1) Methylmalonic acid (C4H6O4), (2) Homocysteine (C4H9NO2S), (3) Xanthurenic acid (C10H7NO4), (4) Pyroglutamic acid (C5H7NO3), (5) Vanilmandelate (C9Hl0O5), (6) 5-Hydroxyindoleacetic acid (C10H9NO3), (7) Quinolinic acid (C7H5NO4), (8) Kynurenic acid (C10H7NO3), (9) Ethylmalonic acid (C5H8O4), (10) Hydroxymethylglutarate (C6H8O5), and (11) 3-hydroxypropyl mercapturic acid (3-HPMA) (C8H15NO4S). In one aspect, the biomarker profile may include measurement of all 11 biomarkers, or 10 biomarkers, or at least 9, or at least 8, or at least 7, or at least 6, or at least 5, or at least 4, or at least 3, or at least 2 biomarkers.

Elevated Methylmalonic acid can indicate a Vitamin B12 deficiency. Patients whose biomarker profile includes a Methylmalonic acid level >2.3 μg/mg may have a Vitamin B12 deficiency which may be contributing to their peripheral neuropathy chronic pain symptoms. Administration of Vitamin B12 in the form of Methylcobalamin may correct the elevated level of Methylmalonic acid and lower the patient's composite biomarker score.

Elevated Xanthurenic acid can indicate a Vitamin B6 deficiency. Patients whose biomarker profile includes a Xanthurenic acid level >0.63 μg/mg may have a Vitamin B6 deficiency which may be contributing to their peripheral neuropathy chronic pain symptoms. Administration of Vitamin B6 in the form of Pyridoxal-5-Phosphate may correct the elevated level of Xanthurenic acid and, thus, lower the patient's composite biomarker score.

Elevated Pyroglutamate can indicate a glutathione deficiency. Patients whose biomarker profile includes a Pyroglutamate level >40 μg/mg may have a glutathione deficiency which may be contributing to their chronic pain symptoms. Administration of the glutathione precursor N-Acetylcysteine (NAC) may correct the elevated level of Pyroglutamate and, thus, lower the patient's composite biomarker score.

Elevated Quinolinic acid and Kynurenic acid can indicate systemic inflammation. Patients whose biomarker profile includes a Quinolinic acid level >6.3 μg/mg and/or a Kynurenic acid level >2.0 may have cytokine mediated systemic inflammation which may be contributing to their chronic pain symptoms. Administration of Curcumin C3, Magnesium (in the form of Magnesium Glycinate) and Vitamin B3 (in the form of Nicotinamide) may correct the elevated levels of Quinolinic acid and Kynurenic acid and, thus, lower the patient's composite biomarker score.

Elevated 3-HPMA can indicate increased acrolein exposure. Patients whose biomarker profile includes a 3-HPMA level >2.4 μg/mg may have increased acrolein exposure which may be contributing to their chronic pain symptoms. Administration of the glutathione precursor N-Acetylcysteine (NAC) may correct the elevated level of 3-HPMA and, thus, lower the patient's composite biomarker score.

Elevated Ethylmalonic acid can indicate a camitine deficiency. Patients whose biomarker profile includes an Ethylmalonic acid level >6.3 μg/mg may have a camitine deficiency which may be contributing to their chronic pain symptoms. Administration of the Camitine (in the form of Acetyl-L-Camitine) may correct the elevated level of Ethylmalonic acid and, thus, lower the patient's composite biomarker score.

Elevated Hydroxymethylglutarate can indicate a Coenzyme QlO deficiency. Patients whose biomarker profile includes a Hydroxymethylglutarate level >5.1 μg/mg may have a Coenzyme QlO deficiency which may be contributing to their chronic pain symptoms. Administration of Coenzyme QlO may correct the elevated level of Hydroxymethylglutarate and, thus, lower the patient's composite biomarker score.

Elevated Homocysteine can indicate a Vitamin B6, Vitamin B12 and/or Folate deficiency. Patients whose biomarker profile includes a Homocysteine level >1.3 μg/mg may have a B vitamin deficiency which may be contributing to their chronic pain symptoms. Administration of Anhydrous Betaine and/or Vitamin B, Vitamin B12 and/or Folate may correct the elevated level of Homocysteine and, thus, lower the patient's composite biomarker score.

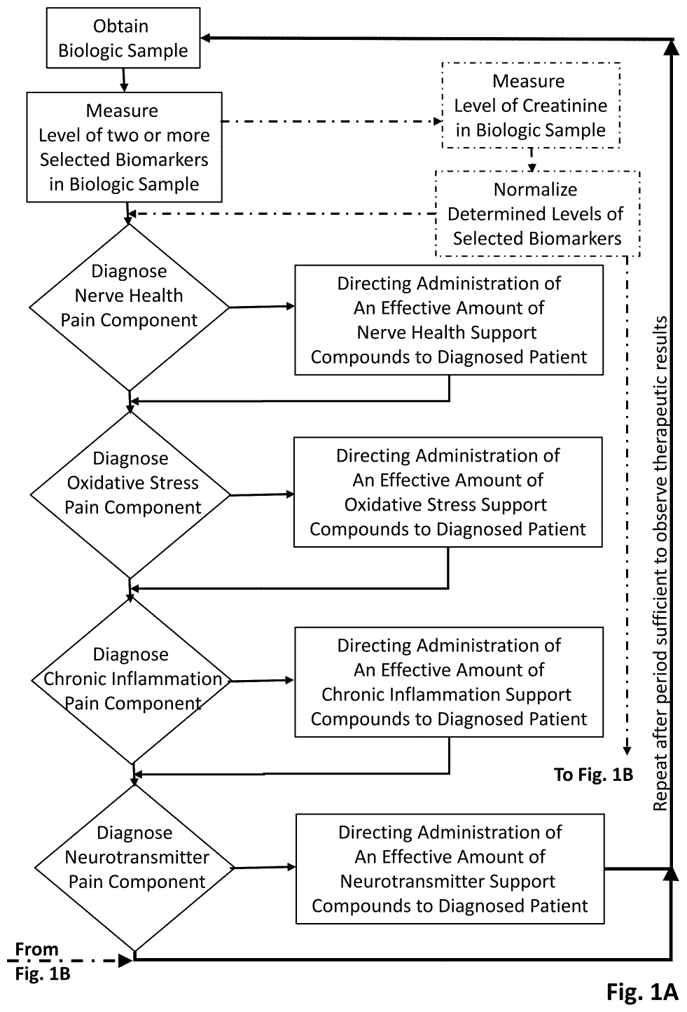

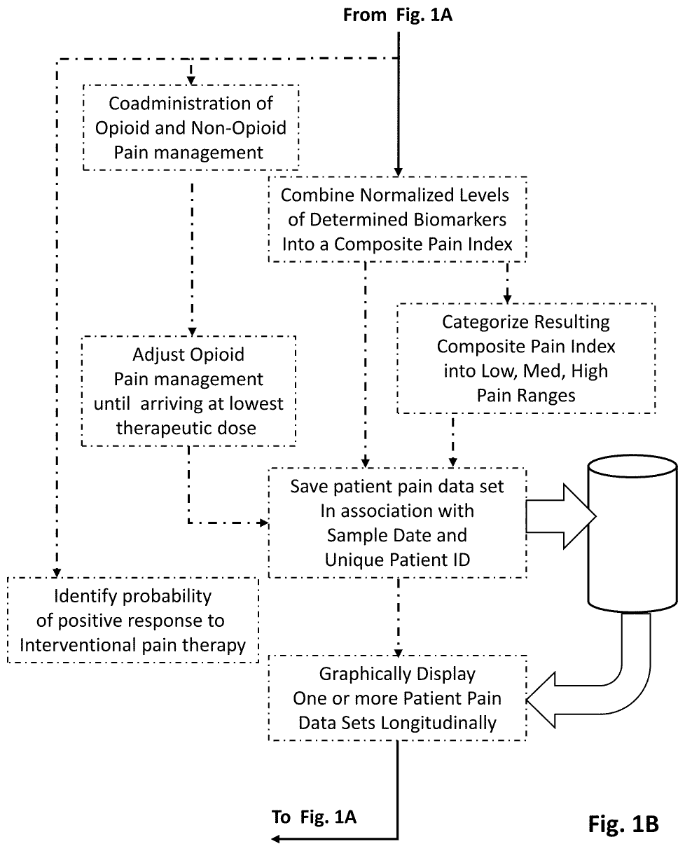

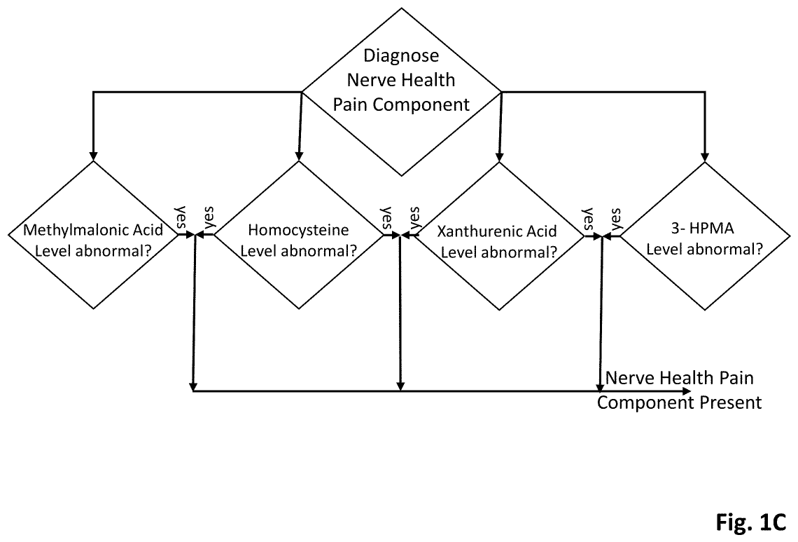

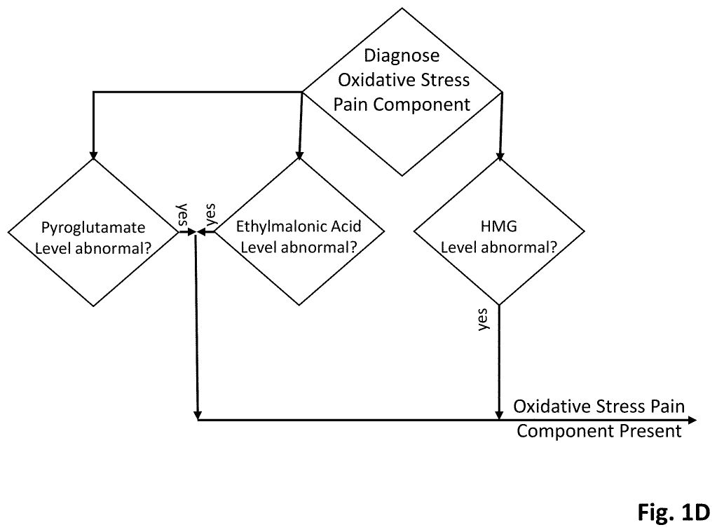

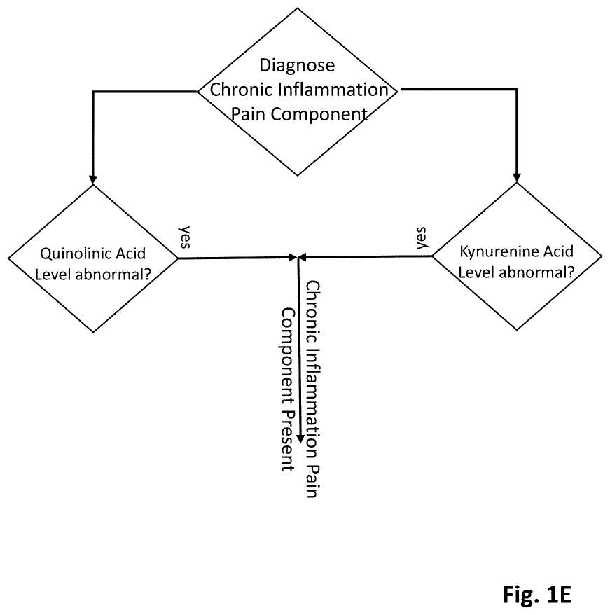

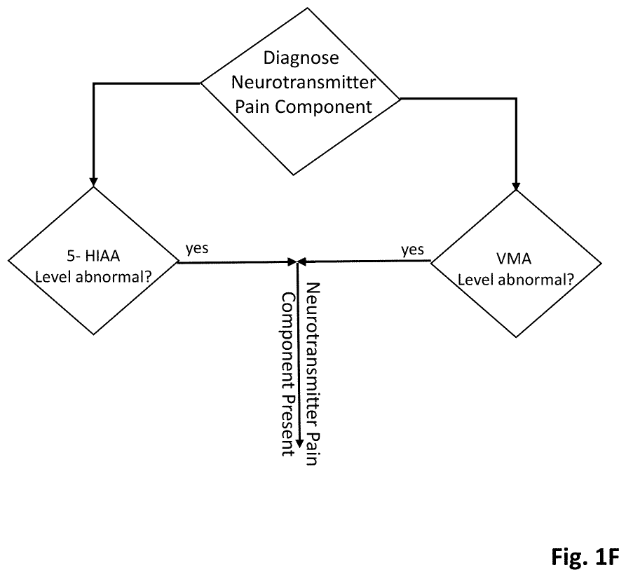

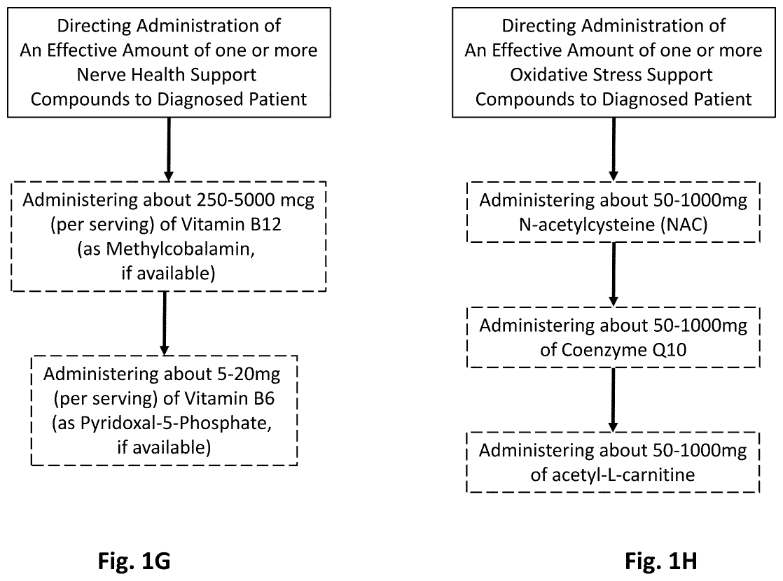

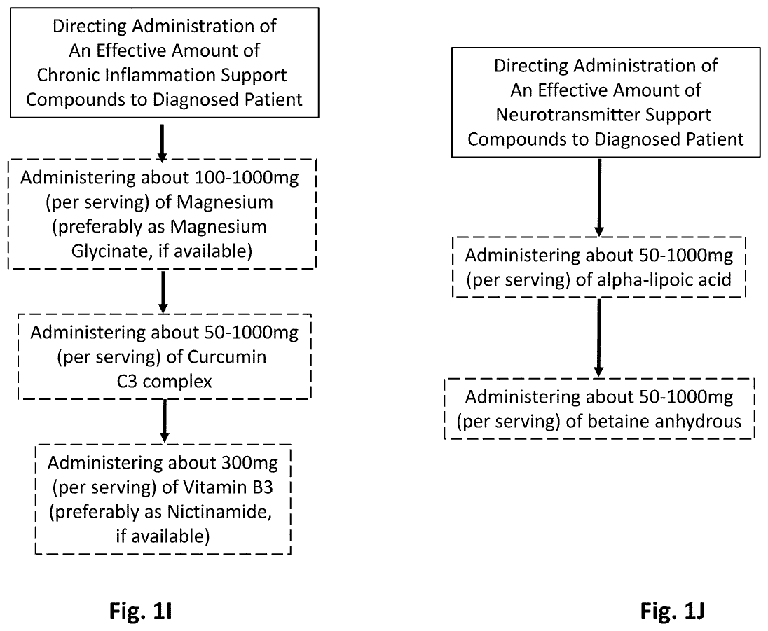

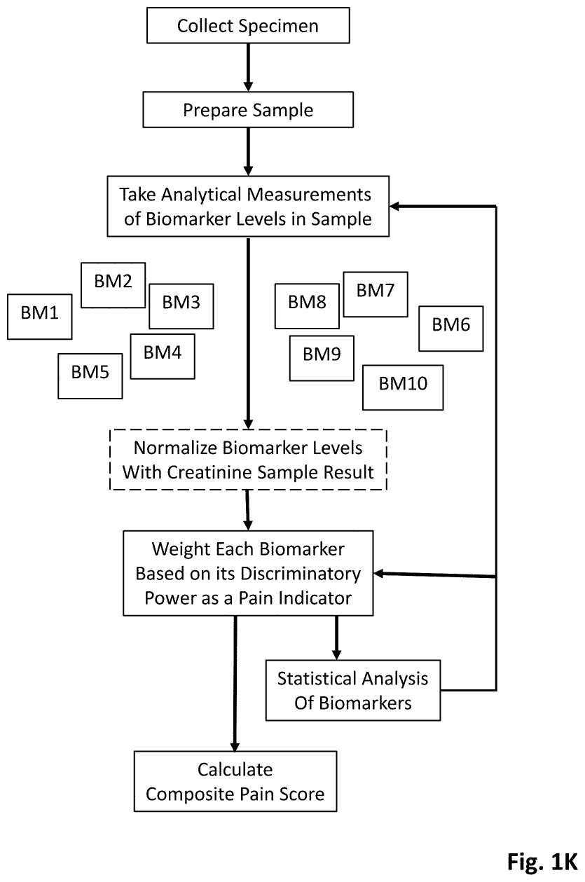

FIGS. 1A, 1B, 1C, 1D, 1E, 1F, 1G, 1H, 1I, 1J and 1K collectively illustrate various potential aspects of a method of diagnosing and treating distinct biologic components contributing to chronic pain experienced by a patient. Starting with FIG. 1A, the basic process of diagnosing and treating the distinct biologic components that contribute to chronic pain experienced by a patient is illustrated. In particular, the method comprises first obtaining a biologic sample from the patient experiencing chronic pain on a sample date (e.g., Jan. 27, 2019). This date may reflect the date the biologic sample (e.g., blood, urine, sweat) was collected from the patient or it may reflect the date the sample was received by the testing laboratory. All that matters is that the date used be utilized consistently used across the system. Then, the process measures the levels of each of two or more biomarkers found in the biologic sample. The two or more biomarkers may be selected from the group comprising: methylmalonic acid, homocysteine, xanthurenic acid, 3-hydroxypropyl mercapturic acid (3-HPMA), pyroglutamate, hydroxymethylglutarate (HMG), quinolinic acid, kynurenine acid, 5-hydroxyindoleacetate (5-HIAA), vanilmandelate (VMA), and ethylmalonic acid.

The process then uses the two or more biomarker levels measured in the biologic sample to diagnose whether the patient has any biochemical abnormalities associated with chronic pain that are sufficiently abnormal to warrant diagnosis and subsequent treatment. For instance, it may be diagnosed based on a measured abnormality in the biochemistry of the sample that a patient has a nerve health pain component to their chronic pain symptoms. This would be based upon a determination that the biologic sample was measured to have an abnormal level of one or all of: methylmalonic acid, homocysteine, xanthurenic acid, and 3-hydroxypropyl mercapturic acid (3-HPMA). Based on findings associated with these potential anomalies, the process should then direct the administration of an effective amount of one or more nerve health support compounds to the patient diagnosed as having the nerve health pain component to their chronic pain symptoms.

Similarly, the process may further determine (i.e., diagnose) from the chemistry of the biologic sample that the patient has an oxidative stress pain component to their chronic pain symptoms. This would be based upon a determination that the biologic sample has an abnormal level of one or all of pyroglutamate, ethylmalonic acid, and hydroxymethylglutarate (HMG) and then then directing the administration of an effective amount of one or more oxidative stress support compounds to the patient diagnosed as having the oxidative stress pain component to their chronic pain.

Similarly, the process may further determine (i.e., diagnose) from the biochemistry of the biologic sample that the patient has a chronic inflammation pain component to their chronic pain. This would be based upon a determination that the biologic sample has an abnormal level of one or both of quinolinic acid and kynurenine acid and then directing the administration of an effective amount of one or more chronic inflammation support compounds to the patient diagnosed as having the chronic inflammation pain component to their chronic pain.

Similarly, the process may further determine (i.e., diagnose) from the biochemistry of the biologic sample that the patient has a neurotransmitter pain component to their chronic pain. This would be based upon a determination that the biologic sample has an abnormal level of one or both of 5-hydroxyindoleacetate (5-HIAA) and vanilmandelate (VMA) and then then directs the administration of an effective amount of one or more neurotransmitter support compounds to the patient diagnosed as having the neurotransmitter pain component to their chronic pain.

In one aspect, individual biomarkers may provide an indication of abnormal biological processes including, but not limited to chronic inflammation, oxidative stress, nerve health and neurotransmitter synthesis/metabolism.

In one aspect, the foregoing references to determining that a biologic sample has an abnormal level of a particular biomarker is determined with reference to 95% double sided, confidence intervals (“C.I.”) that may be used to represent the normal ranges for a population of individuals that have no history of chronic pain or opioid use. 95% double sided confidence intervals may be established by analyzing samples from pain-negative, opioid-free subjects and determining mean values and standard deviations for each of the unique biomarkers. (Here, opioid-free refers to a patient that may have used a short-course of opioids following surgery or an injury at least many months, if not years before the testing.) 95% confidence intervals may be calculated using the equation:

$$X \pm Z \frac{s}{\sqrt{( n )}}$$

Where, X is the mean quantitative value for an individual biomarker
Z is the Z value (1.960)
S is the standard deviation
N is the number of samples obtained and analyzed from the pain-negative cohort

In one aspect, quantitative levels of biomarkers that fall outside this 95% confidence internal (either above the upper limit or below the lower limit) are considered to be abnormal or atypical. In this aspect, the identified abnormal or atypical biomarker values may serve as an opportunity for diagnosis of one or more components associated with a patient's chronic pain symptoms, which in turn, may lead to the direction to administer particular compounds that are indicated may lead to a correction of the abnormal biomarker value and, thus potentially address one or more underlying causes of pain in the individual having symptoms of chronic pain.

In one aspect, the individual levels of each of the metabolomics biomarkers may be higher than those compared to pain-negative levels.

In one aspect, normal biomarker profiles may be attained from a population of pain-negative subjects who have no history of chronic pain symptomology or opioid use by analyzing at least one biological fluid and quantitating levels of metabolomics biomarkers.

In one aspect, pain-positive biomarker profiles may be attained by analyzing at least biological fluid sample from well characterized pain-positive patients who are currently being treated for chronic pain with opioid analgesics.

In one aspect, statistical modeling and chemometric methods will be employed to weight each metabolomic biomarker such that biomarkers which possess the greatest discriminatory power between pain-positive and pain-negative cohorts will be weighted more heavily to contribute to the composite biomarker score than those biomarkers with low discriminatory power.

In one aspect, individual weightings will be determined for each of the biomarkers by comparing biomarker levels from pain-negative cohorts with those from pain-positive cohorts.

In one aspect, pain-free composite biomarker scores may be determined by assessing individual biomarker levels from pain-negative subjects and associated statistical weightings to establish a range of pain-free composite biomarker scores.

In another aspect, pain-positive composite biomarker scores may be calculated using individual biomarker levels and associated statistical weightings for a population of chronic pain patients.

Thresholds to indicate likelihoods (e.g. LOW likelihood; MODERATE likelihood; MODERATELY HIGH likelihood; HIGH likelihood) of atypical biochemical function as a pain determinant may be established using composite scores obtained from both pain-negative and pain-positive cohorts.

In one aspect, patients whose biomarker levels are most different and abnormal compared to pain-free subjects are assigned higher composite biomarker scores, which indicates a greater likelihood that atypical findings could be a cause or contributing factor to chronic pain.

In one aspect, patients whose biomarker levels and composite biomarker scores are very similar to those from the pain-negative subjects are assigned lower composite biomarker scores, indicating a lower likelihood that biochemistry is a cause or contributing factor to chronic pain.

In one aspect, composite biomarker scores indicating likelihood of biochemistry as a pain determinant may range from O to 100, where a composite biomarker score in the range of <20 may represent a LOW likelihood; a composite biomarker score of 20-49 may indicate a MODERATE likelihood; a composite biomarker score of 50-79 may indicate a MODERATELY HIGH likelihood and a composite biomarker score of 80-100 may indicate a HIGH likelihood that abnormal biochemistry is a cause of pain. The end points of these ranges may be adjusted within various systems to account for, among other considerations varying patient populations and standards of care.

In particular, patients with low composite biomarker scores (e.g. less than about 20) may be assigned a LOW likelihood that biochemistry is driving their painful symptoms as their biomarker profile and resultant composite score is similar to pain-negative subjects. Patients assigned this status may be optimally treated with opioid pain medication.

In particular, patients with composite biomarker scores (>20) may be assigned a likelihood (e.g., moderate, moderately high, high) based on the quantitative score as their biomarker profile and resultant composite score is significantly different and higher than those observed in the pain-negative subjects. Patients assigned a particular status may benefit from treatment with a correctional composition as disclosed herein. Those same patients may also additionally benefit from treatment with opioid and/or non-opioid pain medications. Patients assigned a status of “moderately high” or “high” may be benefit from treatment with one or more compounds, which may be combined together into a correctional composition as disclosed herein. Some patients may be best treated without the use of opioid pain medication or with reduced opioid pain medication.

Composite biomarker scores may be determined by adding the individual biomarker scores obtained by comparing patient biomarker results with pain-negative derived 95% confidence intervals and applying appropriate pre-determined weightings. In particular, composite scores are preferably generated by the following summation function of biomarker levels (more preferably when abnormal_concentrations are observed):

$$( {\sum }_{i = 1}^{12} ( {x}_{i} {y}_{i} + 5 ) + ( if {x}_{i} > ( 4 * upper 95 \% CI limit ) , then + 5 ) )$$

Where xᵢ refers to biomarker concentrations (ug/mg, as normalized by [creatinine]), yᵢ refers to statistically validated weighting, and 95% CI refers to the confidence intervals determined for each metabolite. In one embodiment, the following weighting and confidence intervals have been found desirable:

| i | xᵢ | yᵢ | 4*95% CI |
|---|---|---|---|
| 1 | [MMA] | 2 | 9.2 |
| 2 | [HCYS] | 2.1 | 5.2 |
| 3 | [XAN] | 5 | 2.52 |
| 4 | [3-HPMA] | 1.9 | 9.6 |
| 5 | [PGA] low | (1/x)*1.8 | n/a |
| 6 | [PGA] high | 0.2 | 160 |
| 7 | [VMA] | (1/x)*2 | 16.8 |
| 8 | [5-HIAA] | (1/x)*2.3 | 39.2 |
| 9 | [QA] | 1.1 | 25.2 |
| 10 | [KYNA] | 2 | 8.0 |
| 11 | [EMA] | 0.8 | 25.2 |
| 12 | [HMG] | 1 | 20.4 |

In one aspect, the composite biomarker score may be used to categorize an individual as not likely to have atypical biochemistry that contributes to chronic pain or low likelihood of having atypical biochemistry that contributes to chronic pain. In this aspect, the individual may be treated with an opioid.

In one aspect, when the composite biomarker score is used to categorize an individual as having a moderate likelihood of having atypical biochemistry that contributes to chronic pain, a moderately high likelihood of having atypical biochemistry that contributes to chronic pain, or a high likelihood of having atypical biochemistry that contributes to chronic pain, the individual may be treated with an alternative treatment for pain, for example, a treatment that is not an opioid. Alternative treatments for pain will be readily appreciated by one of skill in the art, and include, for example, analgesics, NSAID-type medications, intervention pain therapies, physical therapy, or the like. A further alternative treatment may include a correctional therapy that includes one or more of the compounds/actives found in the compositions described herein.

One approach to a process addressing the elements described above has been illustrated in FIGS. 1A, 1B, 1C, 1D, 1E, 1F, 1G, 1H, 1I, 1J and 1K with the understanding that one of ordinary skill in the art having the present specification and claims before them would understand the illustrated process as well as similar processes that would provide equivalent treatment and opportunities to gather data on the biologic mechanisms of chronic pain, treatments to mediate the sub-components of chronic pain, and any resulting reduction in the patient's need for opioid pain therapy. In particular, these figures disclose that biologic samples may be measured for one or more of biomarkers. The measured levels may be normalized by measuring the level creatinine in the biologic sample and adjusting the measured biomarkers, as may be necessary. The levels of those one or more biomarkers compared to “normal” levels of each biomarker as established by statistical analysis of biomarkers exhibited by pain-negative (and, in some cases, well characterized pain-positive patients who are currently being treated for chronic pain with opioid analgesics). With the information regarding abnormal biomarkers in hand, the patient may be directed to take one or more compounds that support improved functionality of a mechanism that may be associated with a biomechanism that is believed to be contributing to the patient's symptoms of chronic pain (based on the results of diagnostics) in an effort to try to treat some or all of the abnormalities indicated by the patient's abnormal biochemistry. The normalized biomarker levels may be combined into a composite pain index. The calculated result of the composite pain index may be characterized into “Low” “Med” “High” and similar categories. Then, the results and/or composite pain index may be saved in association with the patient's unique ID. The results and/or composite pain index (i.e. a patient pain data set) may also be graphically displayed. This graphic display may also include the option to view longitudinal results of the same data. The ability to review normalized longitudinal results will provide health care providers a means for quantifying components of a patient's chronic pain symptoms. In this way, if the quantities of the composite and/or the respective components change, the patient and their healthcare providers could recognize the differences (and similarities) and determine whether the approach to treatment should be adjusted to address the changes in the scores/values. For instance, providing some measure of discriminatory power would allow professionals to assess changes in a patient with chronic pain symptoms and adjust treatment accordingly. It is believed that one likely adjustment will be the reduction in opioid dosing and thus opioid dependency and the ultimate risk of opioid overdose.

As illustrated in FIGS. 1G-1J, patients may be directed to self-administer effective amounts of various compounds depending upon the nature and extent of the patient's abnormal biomarker results. The types of compounds and amounts (e.g., mcg) used may depend upon a variety of factors, including but not limited to, the extent to which the biomarker values are abnormal. The type and amount (e.g., mcg) of compounds may be changed through a course of treatment as results are collected and measured multiple times over a sufficient span of time to see at least some signs of improvement.

FIG. 1K provides a more specific illustration of one potential approach to measuring and diagnosing potential biologic components contributing to a patient's chronic pain symptoms. In particular, FIG. 1K illustrates measurement of ten biomarkers, which biomarkers may be normalized to accommodate variations in a patient's creatinine levels. Each of the ten biomarkers are weighted such that the biomarkers that provide greater discriminatory power with respect to pain are given a heavier weighting and those biomarkers that provide less discriminatory power with to pain are given lower weighting. The heavier weighted biomarkers provide a greater contribution of the overall composite pain score. In this approach, the composite pain score is calculated from the creatinine-normalized, weighted biomarkers.

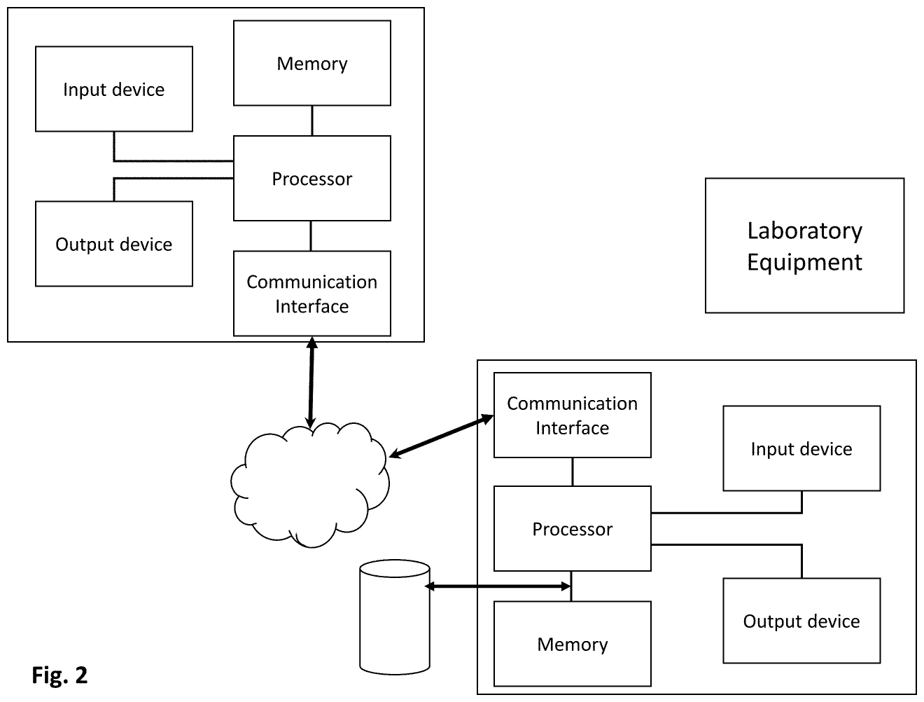

FIG. 2 is a diagrammatic view of the hardware for an exemplary user device and for an exemplary server device (co-located with laboratory equipment) wherein the exemplary user device and exemplary server device are operably connected to one another via a network for use in the method of diagnosing and treating distinct biologic components contributing to chronic pain experienced by a patient. The network may be almost any type of network. For example, in some embodiments, the network may be a version of an Internet network (e.g., exist in a TCP/IP-based network). In some embodiments, the network may be a local area network (such as may be found within a hospital) or a wide area network (such as may be found within a health system with multiple locations). The network may include both wired and wireless portions. The wired portion may use electrical cable, but it may also or alternatively use optical cabling. It is conceivable that in the near future, embodiments within the present disclosure may use more advanced networking technologies.

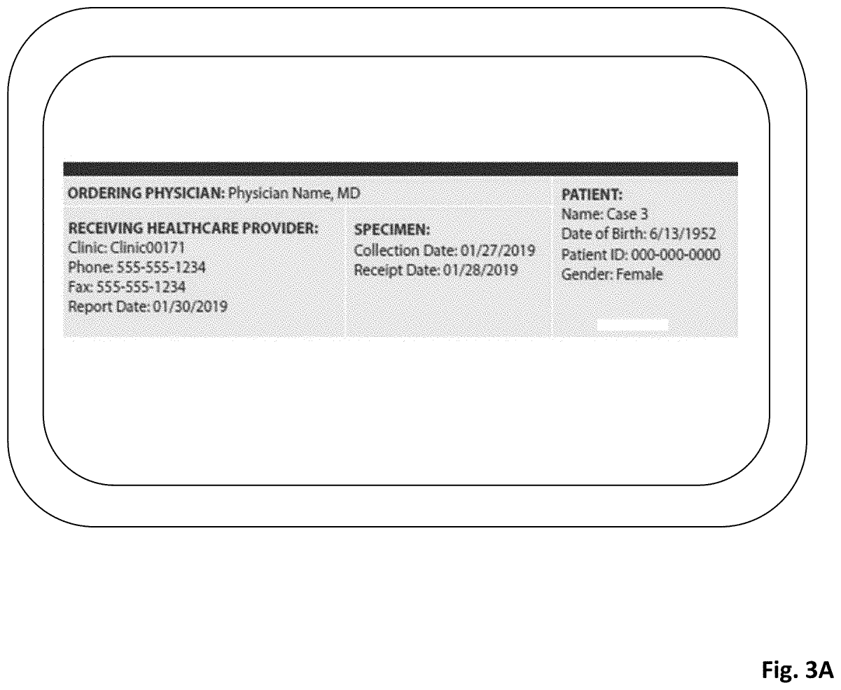

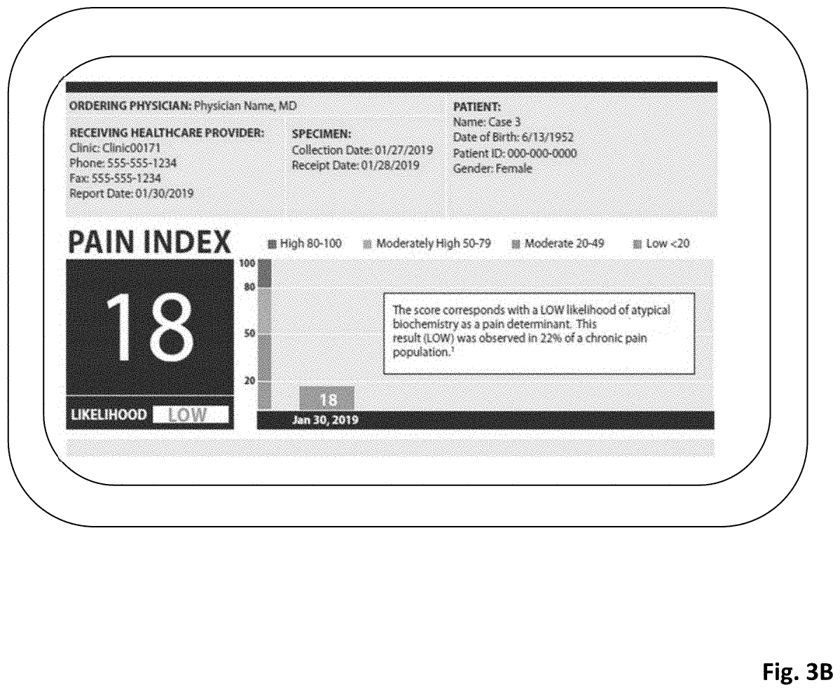

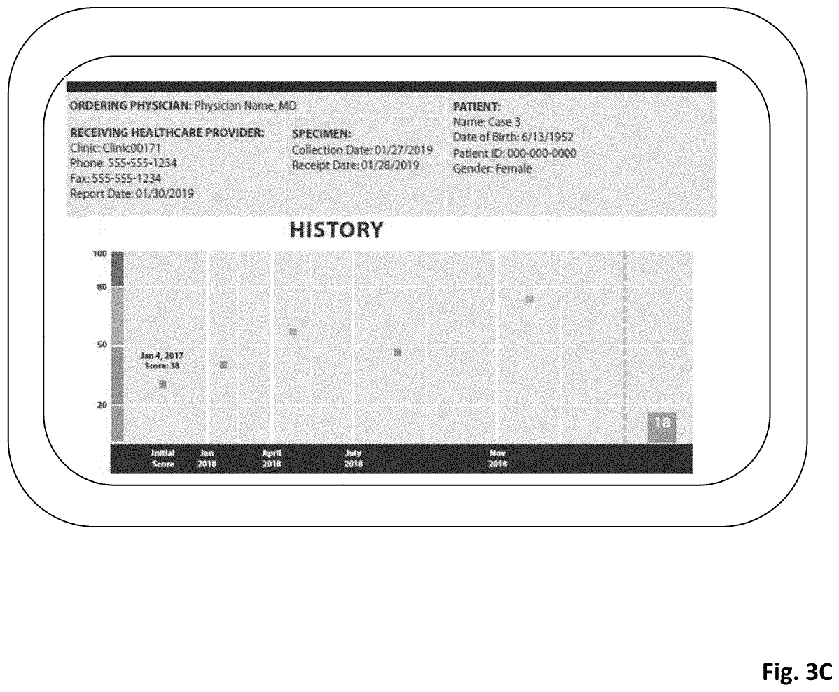

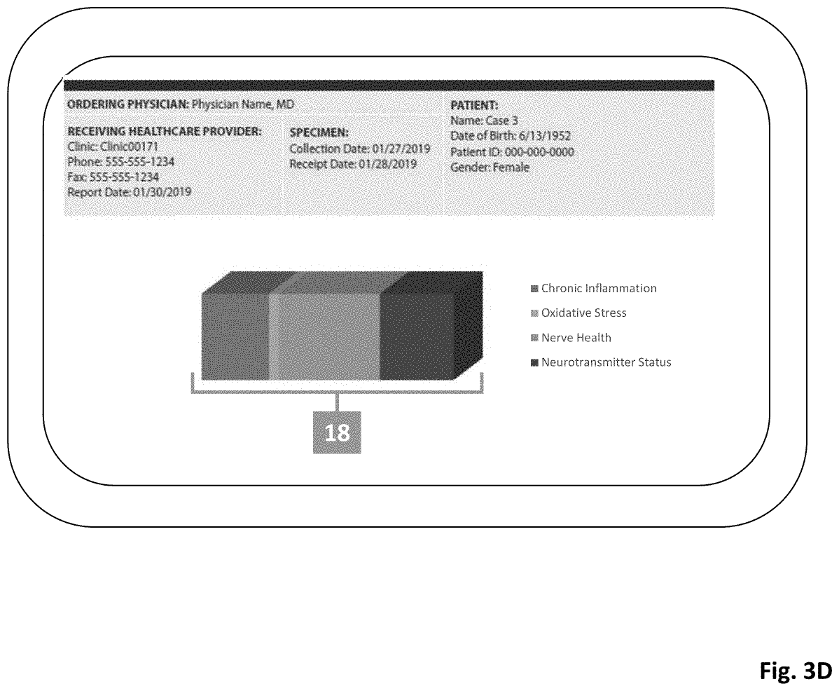

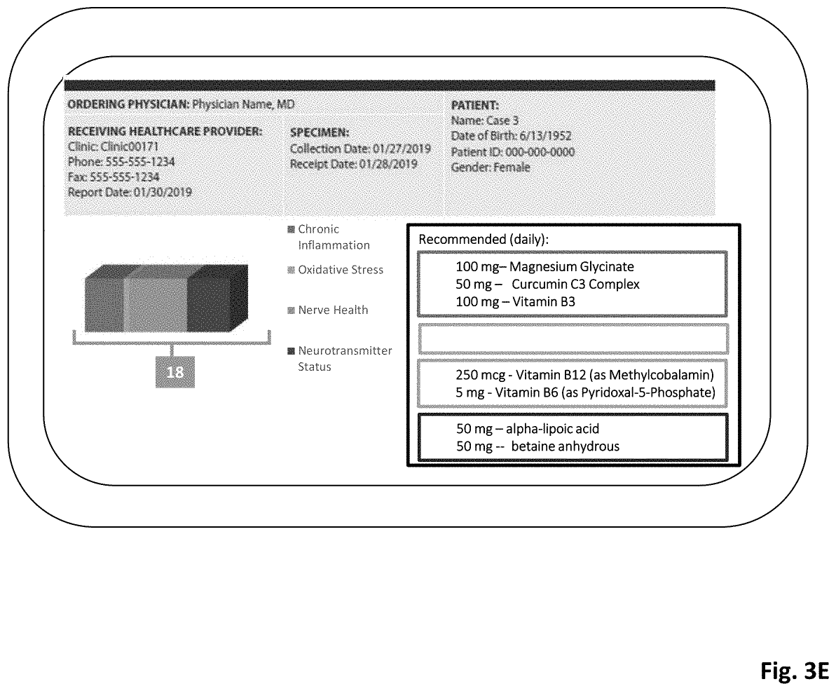

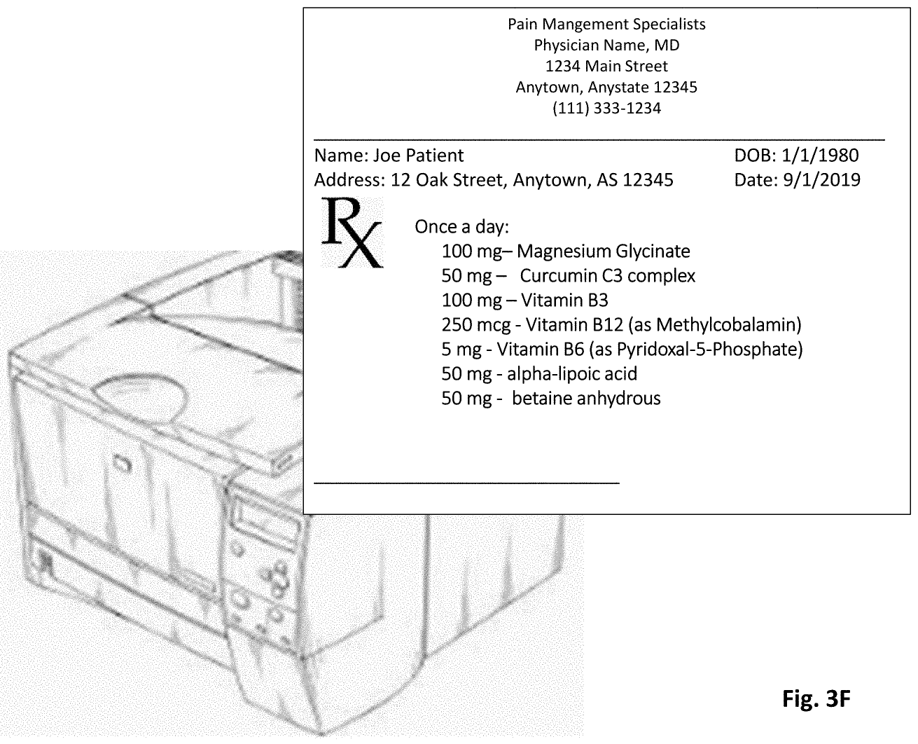

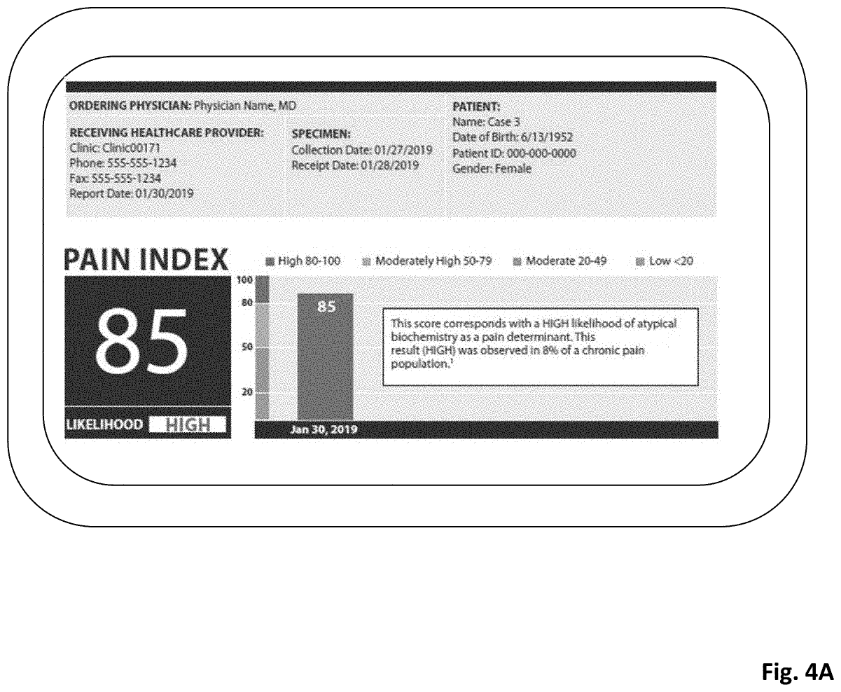

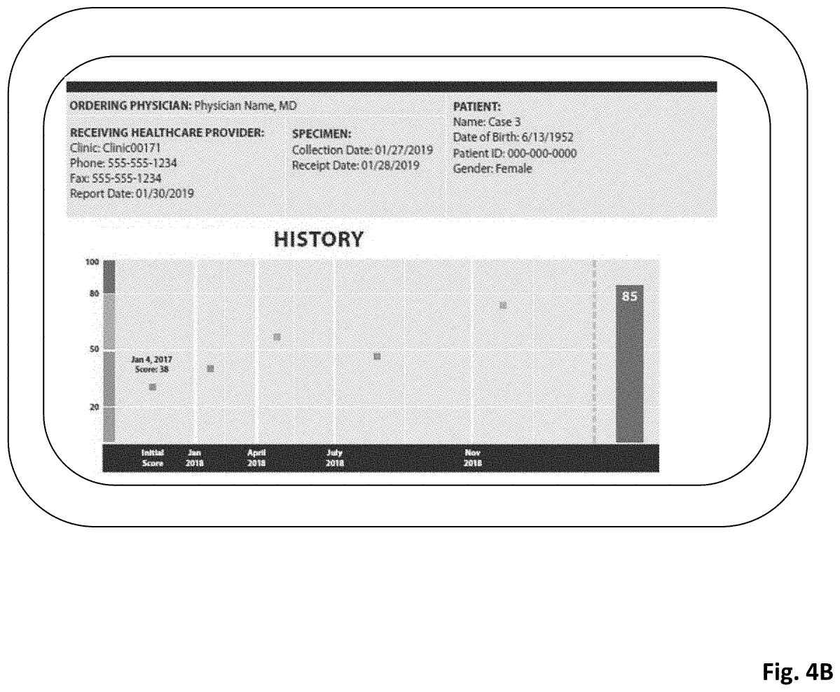

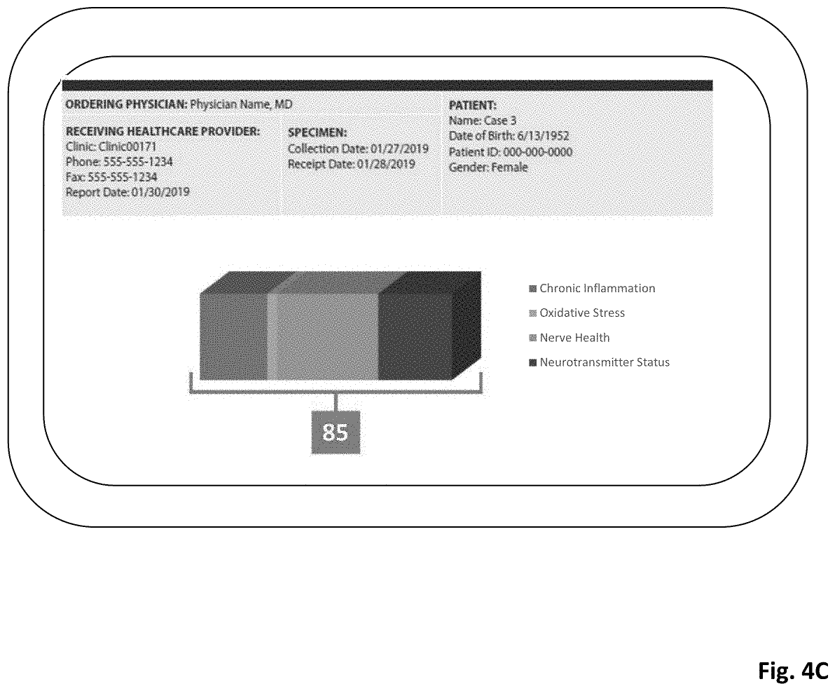

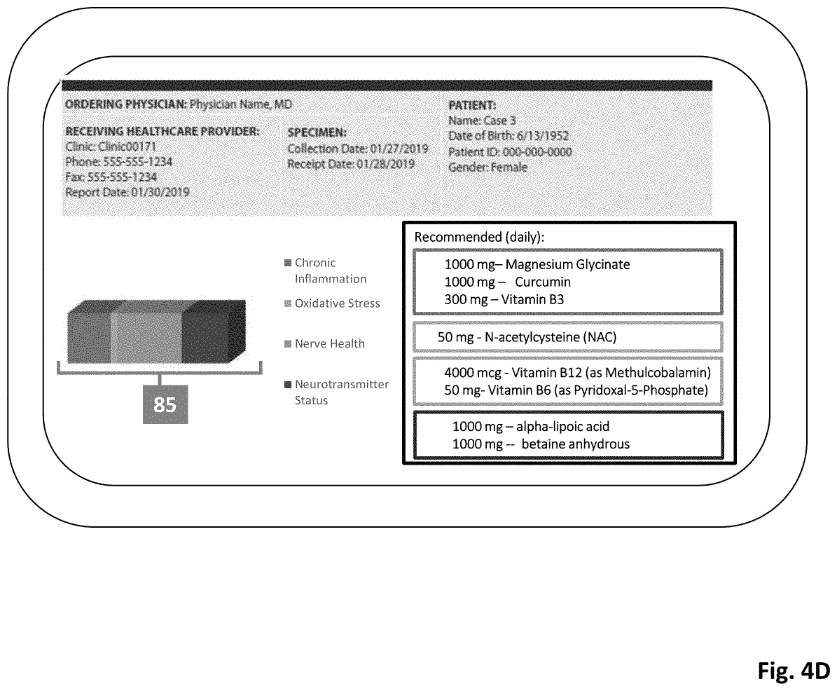

In some embodiments, the exemplar user device may include one or more input devices, one or more output devices (e.g., monitor, touchscreen), one or more processors, one or more communication devices capable of interfacing with the network, one or more non-transitory memory storing processor executable code and/or software application(s), for example including, a web browser capable of accessing a website and/or communicating information and/or data over a wireless or wired network, and/or the like. The memory may also store an application that, when executed by the processor causes the exemplary user device to provide the graphical user interface illustrated in FIGS. 3 and 4.

In particular, FIGS. 3A, 3B, 3C, 3D, 3E, and 3F illustrate various aspects of an exemplary graphical user interface associated with the output device of the exemplary user device as well as the output generated for a printer, such as the illustrated laser printer, for a particular patient who exhibited a low composite pain index. Similarly, FIGS. 4A, 4B, 4C, and 4D, illustrate various aspects of an exemplary graphical user interface associated with the output device of the exemplary user device for another particular patient who exhibited a high composite pain index. In all cases, the output device may be a personal computer (e.g. desktop, laptop), tablet, or smartphone.

Returning to FIG. 3A, an exemplary graphical user interface is illustrated for inputting information regarding the healthcare provider(s), patient and specimen. As would be understood by those of ordinary skill in the art, the operating system of any exemplary user device may support the entry and display of such information in a manner associated with the type of output device being used. Such input may involve a touchscreen, a pointing-device (e.g. mouse), keyboard, or any other means for inputting data into the various types of user devices that may be used with the present invention. The information depicted in FIG. 3A is merely intended to be illustrative of the type of information that may be input into the system. As would be understood by those of skill in the art to which the present invention pertains, additional information such as the medical history and current medications list associated with the patient may be input into the system. In particular, it may be important to know whether or not the patient is currently taking opioids, steroids, non-steroidal anti-inflammatories, and other medications that may impact the testing results.

FIGS. 3B and 4A illustrate one exemplary user interface for conveying the composite pain index along with a category for the composite pain index calculated from a patient's one or more biologic samples over time. In FIG. 3B, the composite pain index is illustrated as being “18,” which is categorized in the LOW range. In FIG. 4A, the composite pain index is illustrated as being “85,” which is categorized in the HIGH range. As illustrated in FIGS. 3B and 4A, the user interface may include four categories for composite pain (i.e., high, moderately high, moderate, and low). The illustrated ranges may have different end-points, may be organized into fewer or more categories and the categories may have different designations (e.g., low, med, high).

FIGS. 3C and 4B illustrate one exemplary user interface for conveying the value of a patient's composite pain index over time. This historical (longitudinal) view of a patient's composite pain index may be useful to show the patient their progress over time. This information may also be useful to the medical community in its study of pain and the impact of various treatments over time. As illustrated in FIGS. 3C and 4B, the composite pain scores are graphed against a common Y-axis representing the composite pain index and X-axis representing time. The placement of values on the X-axis need not be proportional to the time-interval between samples, although there may be applications where maintaining time-proportionality may be constructive to the analysis. In such cases, such proportionality may be observed.

FIGS. 3D and 4C illustrate one exemplary graphical depiction of the constituents of a patient's composite pain index. In the illustration, testing is used to quantify four categories of biological processes: chronic inflammation, oxidative stress, nerve health and neurotransmitter synthesis/metabolism, that are believed to make significant contributions to a patient's chronic pain. As illustrated in FIG. 3D, the exemplary patient of FIGS. 3A-3C is illustrated as having particular sub-scores for each of the four categories. As illustrated in FIG. 3D, the exemplary patient has a very low contribution to their composite pain index from the abnormal biochemistry associated with oxidative stress and the largest contribution to the index from the abnormal chemistry associated with nerve health. Similar contributions to the composite pain index are illustrated in FIG. 3D from chronic inflammation and neurotransmitter status. Different contributions are illustrated in FIG. 4C.

FIGS. 3E and 4D illustrate one exemplary graphical depiction of the recommendations associated with particular contributions of abnormal biochemistry associated with the four categories of biological processes that are believed to contribute to chronic pain. In this illustration, support compounds believed to improve biological processes contributing to particular biological processes contributing to particular categories of chronic pain are color-coded to the categories of biologic processes determined to contribute to the patient's chronic pain. In particular, FIG. 3E illustrates that the exemplary patient has contributions to their chronic pain from chronic inflammation, never health, and neurotransmitter status abnormalities and, in turn, those index values lead to the recommendation of particular amounts of one or more support compounds (believed to be effective in treating a particularly diagnosed biochemical abnormality).

### Compositions

In one aspect, a combination of active agents per Table 6 or 7, below, may be administered in a dosage form selected from intravenous or subcutaneous unit dosage form, oral, parenteral, intravenous, and subcutaneous. In some aspects, active agents provided herein may be formulated into liquid preparations for, e.g., oral administration. Suitable forms include suspensions, syrups, elixirs, and the like. In some aspects, unit dosage forms for oral administration include tablets and capsules. Unit dosage forms configured for administration once a day; however, in certain aspects it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose may be useful because it is readily and economically available and is easy to work with. Other suitable thickening agents include, for example, xanthan gum, carboxymethyl cellulose, hydroxypropyl cellulose, carbomer, and the like. In some aspects, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some aspects, each tablet or capsule contains from about 1 mg or less to about 1,000 mg or more of an active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some aspects, tablets or capsules are provided in a range of dosages to permit divided dosages to be administered. A dosage appropriate to the patient and the number of doses to be administered daily may thus be conveniently selected. In certain aspects two or more of the therapeutic agents may be incorporated to be administered into a single tablet or other dosage form (e.g., in a combination therapy); however, in other aspects the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as com starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or alginic acid or salts thereof.

Binders may be used to form a hard tablet. Binders include materials from natural products such as acacia, tragamayth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzalkonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents. In a preferred embodiment, the active support agents may be formulated into a gel form and packaged in a pouch, such that a patient can rip open the gel pouch and directly ingest the mixture of active support agents.

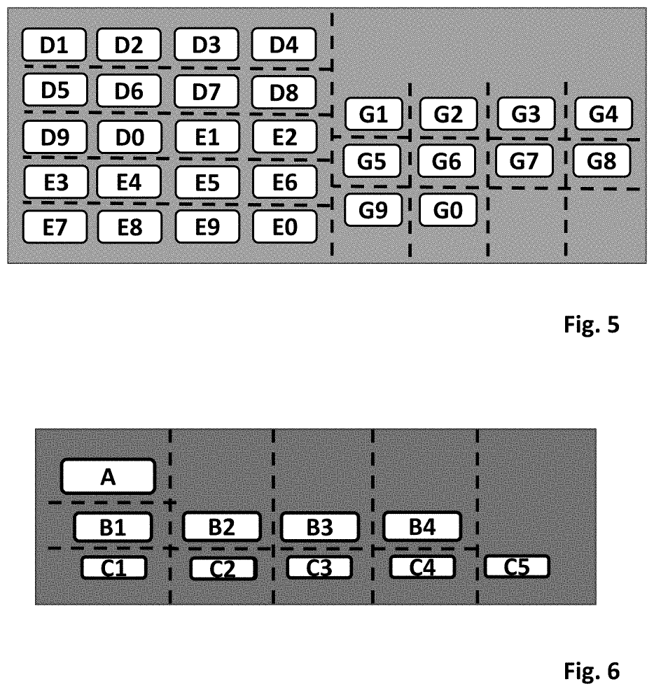

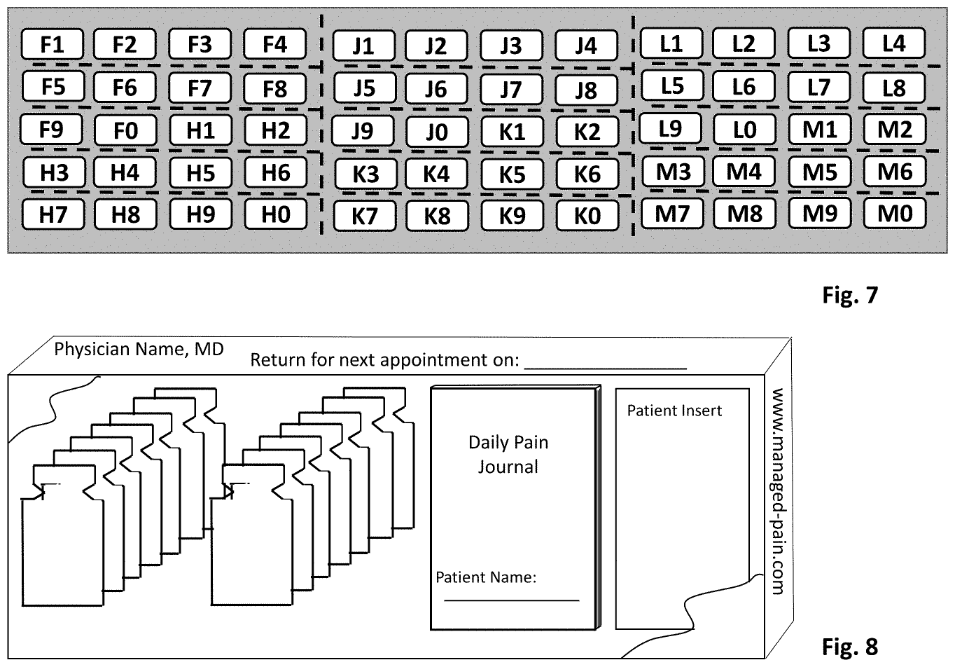

FIGS. 5, 6, and 7 each illustrate novel individually sealed, frangible packaging for a plurality active agents/compounds, e.g., nerve health support compounds, chronic inflammation support compounds, and oxidative stress support compounds, respectively. Each oval represents the smallest unit of a particular compound. For instance, compound D1 may be provided in 200 or 250 mg tablets/capsules and the potentially effective treatment amount may range from 200/250 mg to 5000 mg. As illustrated the packing is color-coded to match the color coding in the report generated on the graphical user interfaces. The packages may be designed so that a healthcare provider, the patient, the patient's family, and/or even a caregiver can separate portions of the package away from the main packing and discard portion of the package that are not needed by the patient. Each package may contain the potential components for the daily dose of the particular biologic support compounds/compositions.

In some aspects, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

In another aspect, the foregoing kits may be distributed to patients in a box that contains—in addition to 7 to 14 days' worth of the active support compound agents and directions for use—a pain diary and satisfaction survey. One of the written materials or box may include a URL that connects the patient with additional content addressing issues of potential interest to patients experiencing chronic pain.

FIG. 8 is a partial cut-away illustrating one potential kit, which comprises a box that contains: (1) a 14 day sample of gel packs containing a particular combination of nerve health support compounds, chronic inflammation support compounds, and/or oxidative stress support compounds; (2) a daily pain journal for patients to chart their experience while trying out the sample gel packs with the support compounds; (3) a patient insert with dosing instructions and other information regarding the compounds in the gel packs as well as a discussion of chronic pain, its components, and benefits of monitoring your composite pain score.

### EXAMPLES

The following non-limiting examples are provided to further illustrate aspects of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific aspects that are disclosed and still obtain a like or similar result without departing from the spirit and scope of the invention.

### Example: Method to Analyze and Quantify Biomarkers

Individual biomarkers may be characterized by mass-to-charge ratio as determined by mass spectrometric techniques including triple quadrupole mass spectrometry (QQQ-MS), Time of flight mass spectrometry (TOF-MS) and single quadrupole mass spectrometry (Q-MS). Biomarkers may be identified and quantified by the shape and area of their spectral peak relative to an internal standard which may be a deuterated form of the analyte of interest. Biomarkers may be characterized and identified according to their binding and retention characteristics to adsorbent surfaces such as chromatographic columns consisting of polar or non-polar solid chemistries with which the biomarker interacts with as it passes through the column on a liquid or gas chromatograph before entering the mass spectrometer. Interaction and binding characteristics and mass-to-charge characteristics provide one method to determine whether a particular detected compound is a biomarker described herein. Spectral, chromatographic and mass-to-charge characteristics represent inherent characteristics of the metabolomic biomarkers and not process limitations in the manner in which the biomolecules are discriminated.

Quantitative measurement of biomarker levels may be carried out using tandem mass spectrometry techniques, preferably selected reaction monitoring (SRM), multiple reaction monitoring (MRM), single ion monitoring (SIM) in combination with liquid chromatography, and/or Enzyme-Linked Immunosorbent Assays (ELISA) for the detection of the compounds.

Biomarker quantification may be achieved using calibration curves which may be prepared with deuterated internal standards and analyte solutions at known concentrations. Biomarkers present in patient samples may be quantified based on their relative signal compared to solutions of known concentration and those of the internal standards which are spiked into both patient samples and calibrators for the purpose of ensuring accurate measurements and eliminating matrix interference or ion suppression.

Individual biomarkers may be unequivocally identified using both retention time characteristics and accurate mass fragmentation patterns which occur in the quadrupole and collision cell components of the mass spectrometer. Individual biomarkers may first be separated from each other by passing the biological sample through a chromatographic column where each biomarker will have unique binding/retention characteristics. Following separation in the chromatographic column, sample constituents may then be introduced to the ionization source of the mass spectrometer where they are ionized to give them an overall positive or negative charge. Once ionized, sample constituents, including all biomarkers present in the sample, may be passed through quadrupoles in order to accurately determine their parent ion mass and fragmentation characteristics that produce daughter or fragment ions. Accurate identification of each biomarker may be made based on chromatographic characteristics such as retention time and peak shape relative to deuterated internal standards. Identification may also be made based on parent ion mass in either positive or negative ionization mode and finally on detection of expected or characteristic daughter or fragment ions at the mass spectrometric detector. Quantification may be achieved using deuterated internal standards which have been prepared at known concentrations and spiked into calibration solutions which are used as part of a calibration curve.

### Example: Sample Preparation Prior to Analysis

Prior to mass spectrometric analysis biological samples obtained from pain-positive and pain-negative cohorts may be subject to sample preparation techniques which aim to enrich, concentrate or extract compounds and classes of biomolecules including biomarkers of interest. Biomarkers and other analytes of interest may be enriched, concentrated or extracted in order to ‘clean up’ the biological sample by removing large interfering biomolecules which are not of interest for the assay described in this invention. Elimination of large compounds, proteins, macromolecules and other complex molecules by enriching, concentration or extracting the compounds of interest may allow for more accurate measurements and reduces the possibility of matrix components interfering with the accurate measurement of desired biomarkers. Biological samples may be enriched, concentrated or prepared for liquid chromatography and mass spectrometric techniques by methods including, but not limited to liquid-liquid extraction (LLE), solid-phase extraction (SPE), simple dilution methods, cation exchange columns, anion exchange columns and multi-mode ion exchange columns.

Deuterated internal standards may be added to patient samples and control samples prior to analysis to ensure accurate measurement/quantitation of individual biomarkers and to eliminate matrix effects or ion suppression phenomena which can greatly impact measurement accuracy.

### Example: Determining Biomarker Weighting

The following Example is provided for a single exemplary analyte, “Biomarker A,” to illustrate the instant invention, and illustrates the methods by which one of skill in the art can readily determine a weighting for carrying out the disclosed methods. Normal range representing the 95% double sided confidence interval for Biomarker A is 20-42 μg/mg which has been calculated using data from a pain-negative cohort and the equation in [069].

Biomarker A weighting is determined based on the discriminatory power that Biomarker A, as a single biomarker, has when distinguishing between the pain-positive cohort and the pain-negative cohort. Biomarkers that have low discriminatory power will have low associated weightings and biomarkers which have strong discriminatory power will have higher weightings. Biomarker A is determined to have a weighting of 0.2.

Biomarker A composite score contribution is calculated by multiplying the severity of the abnormal finding (if any) by the weighting to arrive at a score for this individual biomarker. Patient result for Biomarker A is 49 μg/mg.

Calculation of score contribution is achieved by multiplying the number of units (49 μg/mg) by the weighting (0.2) and added to a constant (+5) to arrive at a score contribution of 14.8.

Patient results that fall within the normal range may contribute 0 to the composite score.

### Example: Low Likelihood of Atypical Chemistry Contributing to Chronic Pain

TABLE 1
Hypothetical example of a composite biomarker score calculation, 95% confidence
interval reference ranges and statistically derived biomarker weightings. A LOW
likelihood result indicates that is a low likelihood that atypical biochemistry
is a cause or contributing cause of pain in this hypothetical patient.

| | Normal | | Biomarker | | |
|---|---|---|---|---|---|
| | Range | Patient | Weighted | Score | |
| BIOMARKER | (95% C.I) | Result | Value | calculation | Likelihood |
| Methylmalonic acid | 0-2.3 | 1.1 | 0 | 0 | |
| Homocvsteine | 0-1.3 | 0.9 | 0 | 0 | |
| Xanthurenic acid | 0-0.63 | 0.41 | 0 | 0 | |
| 3-Hydroxypropyl | 0-2.4 | 0.2 | 0 | 0 | |
| mercapturic acid | | | | | |
| Pyroglutamate (low) | 8-40 | 29 | 0 | 0 | |
| Pyroglutamate (high) | 8-40 | 29 | 0 | 0 | |
| Vanilmandelate | 0.8-4.2 | 3 | 0 | 0 | |
| 5-Hydroxyindoleacetate | 1.6-9.8 | 2.5 | 0 | 0 | |
| Quinolinic acid | 0-6.3 | 8 | 8.8 | 13.8 | |
| Kynurenine acid | 0-2.0 | 1.8 | 0 | 0 | |
| Ethylmalonic acid | 0-6.3 | 6.0 | 0 | 0 | |
| Hydroxymethylglutarate | 0-5.1 | 2.5 | 0 | 0 | |
| Composite Biomarker | | | | 13.8 | LOW |
| Score | | | | | |

### Example: Moderate Likelihood of Atypical Chemistry Contributing to Chronic Pain

TABLE 2
Hypothetical example of composite biomarker score calculations, 95% confidence
interval reference ranges and statistically derived biomarker weightings. A
MODERATE likelihood result indicates that is a moderate likelihood that atypical
biochemistry is a cause or contributing cause of pain in this patient.

| | Normal | | Biomarker | | |
|---|---|---|---|---|---|
| | Range | Patient | Weighted | Score | |
| BIOMARKER | (95% C.I) | Result | Value | calculation | Likelihood |
| Methylmalonic acid | 0-2.3 | 2.9 | 5.8 | 10.8 | |
| Homocysteine | 0-1.3 | 1.7 | 3.57 | 8.57 | |
| Xanthurenic acid | 0-0.63 | 0.58 | 0 | 0 | |
| 3-Hydroxypropyl | 0-2.4 | 0.8 | 0 | 0 | MODERATE |
| mercapturic acid | | | | | |
| Pyroglutamate (low) | 8-40 | 32 | 0 | 0 | |
| Pyroglutamate (high) | 8-40 | 32 | 0 | 0 | |
| Vanilmandelate | 0.8-4.2 | 1.8 | 0 | 0 | |
| 5-Hydroxyindoleacetate | 1.6-9.8 | 6.2 | 0 | 0 | |
| Quinolinic acid | 0-6.3 | 11.5 | 12.65 | 17.65 | |
| Kynurenine acid | 0-2.0 | 3.1 | 6.2 | 11.2 | |
| Ethylmalonic acid | 0-6.3 | 4.1 | 0 | 0 | |
| Hydroxymethylglutarate | 0-5.1 | 3.0 | 0 | 0 | |
| Composite Biomarker | | | | 48.22 | |
| Score | | | | | |

### Example: Moderately High Likelihood of Atypical Chemistry Contributing to Chronic Pain

TABLE 3
Hypothetical example of composite biomarker score calculations, 95% confidence interval
reference ranges and statistically derived biomarker weightings. A MODERATELY HIGH
likelihood result indicates that is a moderately high likelihood that atypical
biochemistry is a cause or contributing cause of pain in this patient.

| | Normal | | Biomarker | | |
|---|---|---|---|---|---|
| | Range | Patient | Weighted | Score | |
| BIOMARKER | (95% C.I.) | Result | Value | calculation | Likelihood |
| Methvlmalonic acid | 0-2.3 | 4.2 | 8.2 | 13.2 | |
| Homocysteine | 0-1.3 | 2.3 | 4.83 | 9.83 | |
| Xanthurenic acid | 0-0.63 | 0.50 | 0 | 0 | |
| 3-Hydroxypropyl | 0-2.4 | 0.2 | 0 | 0 | |
| mercapturic acid | | | | | |
| Pyroglutamate (low) | 8-40 | 42 | 0 | 0 | |
| Pyroglutamate (high) | 8-40 | 42 | 8.4 | 13.4 | |
| Vanilmandelate | 0.8-4.2 | 3 | 0 | 0 | |
| 5-Hydroxyindoleacetate | 1.6-9.8 | 2.5 | 0 | 0 | |
| Quinolinic acid | 0-6.3 | 9.3 | 10.23 | 15.23 | |
| Kvnurenine acid | 0-2.0 | 2.2 | 4.4 | 9.4 | |
| Ethvlmalonic acid | 0-6.3 | 7 | 5.6 | 10.6 | |
| Hydroxymethylglutarate | 0-5.1 | 5.0 | 0 | 0 | |
| Composite Biomarker | | | | 71.66 | MODERATELY |
| Score | | | | | HIGH |

### Example: High Likelihood of Atypical Chemistry Contributing to Chronic Pain

TABLE 4
Example of composite biomarker score calculations using patient test results,
95% confidence interval reference ranges and statistically derived biomarker
weightings. A HIGH likelihood result indicates that is a high likelihood that
atypical biochemistry is a cause or contributing cause of pain in this patient.

| | Normal | | Biomarker | | |
|---|---|---|---|---|---|
| | Range | Patient | Weighted | Score | |
| BIOMARKER | (95% C.I.) | Result | Value | calculation | Likelihood |
| Methylmalonic acid | 0-2.3 | 4.5 | 9 | 14 | |
| Homocysteine | 0-1.3 | 3.2 | 6.72 | 11.72 | |
| Xanthurenic acid | 0-0.63 | 3.0 | 15 | 25 | |
| 3-Hydroxypropyl | 0-2.4 | 3.1 | 5.89 | 10.89 | |
| mercapturic acid | | | | | |
| Pyroglutamate (low) | 8-40 | 35 | 0 | 0 | |
| Pyroglutamate (high) | 8-40 | 35 | 0 | 0 | |
| Vanilmandelate | 0.8-4.2 | 0.4 | 5 | 10 | |
| 5-Hydroxyindoleacetate | 1.6-9.8 | 0.4 | 5.75 | 10.75 | |
| Quinolinic acid | 0-6.3 | 6.0 | 0 | 0 | |
| Kynurenine acid | 0-2.0 | 1.5 | 0 | 0 | |
| Ethylmalonic acid | 0-6.3 | 5.0 | 0 | 0 | |
| Hydroxymethylglutarate | 0-5.1 | 8 | 8 | 13 | |
| Composite Biomarker | | | | 95.36 | HIGH |
| Score | | | | | |

### Example: Treatment of Subjects Using Targeted Intervention and Effect on Composite Score

Table 5. Example highlighting real treatment outcomes on composite biomarker scores. Individuals were tested at baseline and then provided with a targeted nutraceutical formulation, such as the formulation described below, designed to correct abnormal biomarker findings. Subjects took the targeted nutraceutical daily for 30 days before being retested. Post treatment biomarker scores indicate significant improvement in biochemistry, especially in the three subjects (1,3,4) that had the most abnormal findings at baseline.

TABLE 5
Treatment Outcomes for Four Subjects:

| | | | | Post | |
|---|---|---|---|---|---|
| | Baseline | | | Treatment | |
| | Composite | Treatment | Treatment | Composite | |
| Subject | Score | Intervention | Duration | Score | Improvement |
| Subject | 65 | Targeted | 30 days | 26 | 60% |
| 1 | | nutraceutical | | | |
| Subject | 11 | Targeted | 30 days | 11 | 0% |
| 2 | | nutraceutical | | | |
| Subject | 25 | Targeted | 30 days | 2 | 92% |
| 3 | | nutraceutical | | | |
| Subject | 63 | Targeted | 30 days | 10 | 84% |
| 4 | | nutraceutical | | | |

TABLE 6
Active Ingredients of targeted nutraceutical:

| | Amount | |
|---|---|---|
| | per | |
| Ingredient | Serving | Indication |
| Vitamin B3 (as | 300 mg | Elevated Inflammatory markers |
| Nicotinamide) | | (Quinolinic acid, Kynurenic |
| | | acid) |
| Vitamin B6 (as | 20 mg | Elevated Xanthurenic acid |
| Pyridoxal-5-Phosphate) | | (indicative of Vitamin B6 |
| | | deficiency) |
| Vitamin B12 (as | l000 mcg | Elevated Methylmalonic acid |
| Methylcobalamin) | | (indicative of Vitamin B12 |
| | | deficiency) |
| Magnesium (as | 250 mg | Elevated Inflammatory markers |
| Magnesium Glycinate) | | (Quinolinic acid, Kynurenic |
| | | acid) |
| N-Acety1-L-Cysteine | 200 mg | Elevated Pyroglutamate |
| | | (indicative of glutathione |
| | | depletion) |
| Ashwagandha Root | 200 mg | Elevated Vanilmandelate |
| Powder | | (indicative of increased |
| | | epinephrine/norepinephrine |
| | | turnover) |
| Curcumin C3 | 200 mg | Elevated Inflammatory markers |
| Complex | | (Quinolinic acid, Kynurenic |
| | | acid) |
| Alpha-Lipoic acid | 200 mg | Elevated Oxidative Stress |
| | | markers(Pyroglutamate, |
| | | Ethylmalonic acid, |
| | | Hydroxymethylglutarate) |
| Coenzyme Q10 | 200 mg | Elevated Hydroxymethylglutarate |
| | | (indicative of Coenzyme QlO |
| | | depletion) |
| Acety1-L-Camitine | 300 mg | Elevated Ethylmalonic acid |
| | | (indicative of Carnitine |
| | | depletion) |
| Betaine Anhydrous | 250 mg | Elevated Homocysteine |
| | | (indicative of decreased |
| | | homocysteine recycling) |

TABLE 7
Active Ingredients of another potential example of targeted nutraceutical

| | Amount | Amount | Amount | Amount | Amount | Amount |
|---|---|---|---|---|---|---|
| | per | per | per | per | per | per |
| Ingredient | Serving | Serving | Serving | Serving | Serving | Serving |
| Vitamin B3 | 300 mg | 50 mg | 100 mg | 400 mg | 500 mg | 1000 mg |
| (as Nicotinamide) | | | | | | |
| Vitamin B6 | 20 mg | 5 mg | 10 mg | 30 mg | 40 mg | 50 mg |
| (as Pyridoxal-5- | | | | | | |
| Phosphate) | | | | | | |
| Vitamin B12 | 1000 mcg | 100 mcg | 500 mcg | 1250 mcg | 1500 mcg | 2000 mcg |
| (as Methylcobalamin) | | | | | | |
| Magnesium | 250 mg | 25 mg | 100 mg | 500 mg | 750 mg | 1000 mg |
| (as Magnesium | | | | | | |
| Glycinate) | | | | | | |
| N-Acetyl-L-Cysteine | 200 mg | 20 mg | 100 mg | 500 mg | 750 mg | 1000 mg |
| Ashwagandha Root | 200 mg | 20 mg | 100 mg | 500 mg | 750 mg | 1000 mg |
| Powder | | | | | | |
| Curcumin C3 | 200 mg | 20 mg | 100 mg | 500 mg | 750 mg | 1000 mg |
| Complex | | | | | | |
| Alpha-Lipoic acid | 200 mg | 20 mg | 100 mg | 500 mg | 750 mg | 1000 mg |
| Coenzyme Q10 | 200 mg | 20 mg | 100 mg | 500 mg | 750 mg | 1000 mg |
| Acetyl-L-Camitine | 300 mg | 20 mg | 100 mg | 500 mg | 750 mg | 1000 mg |
| Betaine Anhydrous | 250 mg | 20 mg | 100 mg | 500 mg | 750 mg | 1000 mg |

All percentages and ratios are calculated by weight unless otherwise indicated. All percentages and ratios are calculated based on the total composition unless otherwise indicated.

### Example: A Clinical Study

A clinical study to validate the use of mechanistic pain biomarkers to objectively identify perturbed biochemistry contributing to painful symptoms was conducted to confirm the potential impact of such biomarkers on treatment decision making, patient management and simplify a path toward personalized, non-opioid pain management.

The study used the preferred multi-biomarker assay and particular variables with the equations (described above) in diverse cohorts of chronic pain patients. Study subjects were recruited by clinical investigators at sites across the United States and provided a single urine sample after consenting to participate. Levels of 11 urinary pain biomarkers were measured and combined using particular variables with the equations (described above) to generate what shall be referred to as “FPI scores” (ranging from 0-100). Relationships between FPI scores and clinical pain assessments were characterized using Pearson's correlations and area under the receiver operating characteristic curve (AUROC) to discriminate between subjects with low and high FPI scores. These statistical methodologies were also employed to characterize the relationships between FPI scores from the chronic pain cohort and age, sex-matched healthy controls.

The results of this clinical study showed that FPI scores were significantly correlated with SF-36 scores among chronic pain patients with high vs. low FPI (P-value <0.015). The results of this clinical study further showed that FPI scores significantly correlated with specific components of the SF-36 assessment, including emotional well-being, limitations due to emotional problems, and general health (P-value <0.05). Area under ROC analysis (AUROC) on the results of this clinical study revealed the FPI score to accurately distinguish biomarker profiles between healthy and chronic pain cohorts (AUROC: 0.7490, P-value <0.001) as well as the SF-36 scores between chronic pain patients with low vs. high FPI scores (AUROC: 0.7715, P-value <0.01).

The specifics of this clinical study are described hereinbelow.

A total of 154 chronic pain patients were enrolled in the prospective, multi-site clinical study. This study was reviewed and approved by Western Institutional Review Board (WIRB). Informed written consent was obtained upon enrolment and all subjects completed the 36-item short form survey (SF-36) and the Hospital Anxiety and Depression Scale (HADS) at the time of sample collection. A single urine sample was obtained from each subject following enrolment and completion of all necessary surveys/questionnaires. Samples were packaged and shipped to Ethos Laboratories (Newport, Ky.) where they were accessioned, prepared and analyzed according to standard operating procedures.

### Inclusion/Exclusion Criteria

TABLE 8
Description of inclusion/exclusion criteria for clinical study

| Inclusion criteria | Exclusion criteria |
|---|---|
| Men and women between the | Severe or untreated psychiatric |
| ages of 21 and 75 | disturbance |
| Long term use (>6 months) of | Liver and/or kidney disease |
| an opioid analgesic at a current | |
| daily dose of 30 milligram | |
| morphine equivalents (MME) | |
| or greater | |
| Currently under the care of a | Pregnancy |
| participating investigator | |
| Understands and complies with | Use of corticosteroid or another |
| all sample collection procedures | immunosuppressive drug during |
| | or 1 month prior to sample |
| | collection |
| | Diagnosed with bacterial or viral |
| | infection during or 3 months prior |
| | to the study |
| | Being prescribed anti-cytokine |
| | therapies |
| | Use of the following dietary |
| | supplements in the previous 3 |
| | months: B vitamins (B1, B2, |
| | B3, B5, B6, B12); Folate or |
| | Folic acid; Magnesium; |
| | N-Acetyl-Cysteine (NAC); |
| | Ashwagandha; Curcumin or |
| | Turmeric; Alpha-Lipoic acid; |
| | Coenzyme Q10; Carnitine; |
| | Tryptophan or other Amino |
| | acid powder |

### Healthy Control Samples in Clinical Study

Control samples from healthy individuals with no history of chronic pain or opioid use were collected prospectively from Lee Biosolutions (Maryland Heights, Mo.) and their associated clinical collection sites across the United States. Following collection, all healthy control samples were shipped frozen to Ethos Laboratories (Newport, Ky.) where they were thawed, prepared and analyzed according to standard operating procedures.

### Analytical Methodologies in Clinical Study

Samples were accessioned into the Laboratory Information System (LIS) and frozen at −20° C. on the date of receipt and remained frozen until the time they were thawed at room temperature and prepared for analysis. Each sample underwent only one freeze/thaw cycle. Samples were prepared on 96-well plates with four-point calibrators, two quality controls, and one negative sample per plate. The urinary biomarkers and creatinine were each analyzed in separate LC-MS/MS assays. All analytes were quantitated using isotopic dilution and analyzed with MassHunter software. Biomarker concentrations were corrected for urinary dilution using creatinine and normalized values were reported as μg/mg creatinine.

### Calculation of FPI and Score Derivation Used in Clinical Study

With the intent to distinguish between healthy and chronic pain populations, particular variables were tried with the equations (described above) to calculate “FPI score” based on binary logistic regression, where individual biomarkers that deviate from normal concentrations collectively contribute to a decision-making process that sorts samples as either normal (healthy) or abnormal (pain). Various types of supervised machine learning techniques, including principal component analysis, clustering, and linear discriminant analysis (LDA) were compared among randomly selected healthy and chronic pain samples, and LDA proved to provide the most robust segregation between groups (P-value <0.001). This model of multiple analysis of variance (MANOVA) with LDA generated weighted coefficients for each biomarker according to the function below:

Y=β0+β1*E+β2*F+β3*H+β4*I+β5*J+β6*K . . .
where Y refers to the model output, E-K refer to metabolite concentrations as normalized to creatinine levels (ug/mg) in urine, β0 is the y-intercept, and β1-β6 are coefficients generated by analysis that most distinguishes control (healthy) and experimental (chronic pain) groups for each metabolite.

Biomarkers used in the development of the FPI model were transformed for normal distribution by Box-cox transformations (x^{λ}, where λ=lambda, R 3.5.1 programming) and verified by normality analysis (D'Agostino and Pearson Test, GraphPad Prism 8.3.0).

TABLE 9
Values used for Box-cox transformations for Clinical Study Results

| X | Lambda (λ) | X | Lambda (λ) |
|---|---|---|---|
| MMA | 1/log₁₀(x) | QA | 1 |
| HCYS | −0.25 | KYNA | log₁₀(x) |
| XAN | −0.01 | HMG | log₁₀(x) |
| PGA | 1 | EMA | −0.05 |
| VMA | −0.25 | 3-HPMA | −0.5 |
| 5-HIA | 0.8 | | |

### Statistical Methods Used in Clinical Study

To compare univariate patient characteristics between groups, non-parametric t-tests were used to compare means of variables. Multivariate analysis (least squares) were used to determine distinguishable power of the multi-biomarker score. To achieve normal distributions of data prior to MANOVA and correlation analyses, box-cox transformations (λ, lambda) and outlier analyses (determined by >2.5*standard deviation) were applied. Biomarkers are expressed as normalized to creatinine concentrations (μg/mg) for each urine sample. Data was analyzed with level of significance at α=0.05.

Receiver operating characteristics (ROC) and its area under the curve (AUROC) were performed to determine sensitivity and specificity of FPI between healthy vs. pain subjects and low vs. high FPI among pain patient SF-36 scores.

To account for non-normal distribution of clinical assessments among chronic pain patients, Spearman rank's coefficient (r) was used. Correlation analysis was performed for group characteristics (age, sex, creatinine levels, biomarker concentrations) between healthy and chronic pain subjects, as well as among chronic pain patients (biomarker levels, FPI, BMI, daily MME, current VAS, SF-36 score, HADS Anxiety and Depression scores). Components within the SF-36 assessment used for analysis include assessments of physical functioning, limits due to physical health, limits due to emotional problems, energy/fatigue, emotional well-being, social functioning, pain, general health, and health change.

### Results from Clinical Study

Cohort characteristics. FPI scores were calculated using creatinine-normalized biomarker results from 154 chronic pain patients meeting all inclusion criteria and 334 age and sex matched healthy controls with no history of chronic pain or opioid use. The mean age of chronic pain subjects was 55.5 (55.5±11.4) with a slight majority being female (52%). Mean BMI in the chronic pain cohort was 30.9 (30.9±7.57) and 24.8% reported smoking cigarettes. Primary pain diagnoses across the pain cohort included but were not limited to low back pain, lumbar radiculopathy, chronic pain syndrome and cervicalgia and the mean daily dose of opioid medication was 65.3 morphine milligram equivalents (MME) (65.3±42.8). At the time of sample collection, the mean self-reported VAS score was 5.92 (5.92±1.9) while the mean SF-36 score across the pain cohort was 1674 (1674±667). Hospital anxiety and depression surveys revealed a mean anxiety score of 7.12 (7.12±4.11) and a mean depression score of 7.43 (7.43±12.05). Mean component scores from the SF-36 assessment are detailed further in Table 10.

TABLE 10
Characteristics of Pain Patients involved Clinical Study

| Number of subjects | 153 |
|---|---|
| Male (%) | 48 |
| Female (%) | 52 |
| Cigarette smoker (%) | 24.8 |
| | mean ± SD |
| Age | 55.5 ± 11.4 |
| BMI | 30.9 ± 7.57 |
| Daily MME | 65.3 ± 42.8 |
| Current VAS | 5.92 ± 1.9 |
| SF-36 score | 1674 ± 667 |
| HADS A Score | 7.12 ± 4.11 |
| HADS D Score | 7.43 ± 12.05 |

SF-36 score assessment (%)

| | |
|---|---|
| Physical functioning | 45 ± 29 |
| Limits due to physical health | 26 ± 33 |
| Limits due to emotional problems | 61 ± 45 |
| Energy/fatigue | 36 ± 23 |
| Emotional well-being | 67 ± 32 |
| Social functioning | 54 ± 33 |
| Pain | 33 ± 20 |
| General health | 47 ± 26 |
| Health change | 42 ± 26 |

**Patients prescribed long-term opioids (>6 months prescription).

FPI Score Performance and Validation: Abnormal biomarker results were detected in a large majority of the chronic pain cohort with significant deviation from normal ranges observed across most individual biomarkers (see Table 11). Eighty six percent of pain subjects exhibited at least one biomarker, defined as being outside of the double sided, 95% confidence interval reference range. Mean comparisons of individual biomarkers using non-parametric t testing revealed extremely strong discriminatory power (P<0.0001) of five of the eleven biomarkers (Methylmalonic acid, Xanthurenic acid, Pyroglutamate, Kynurenic acid and Hydroxymethylglutarate) while an additional three biomarkers (Homocysteine, Quinolinic acid and 3-HPMA) exhibited statistically significant discriminatory power (P<0.05) between the pain and healthy cohorts. Multi-biomarker statistical models (FPI Score) outperformed individual biomarkers when discriminating between healthy and chronic pain cohorts (AUROC: 0.7490, P<0.0001). Mean FPI scores were evaluated across healthy and chronic pain cohorts and confirm a greater degree of pain-relevant biochemical perturbations in chronic pain subjects (Table 11). Mean FPI scores (0-100 scale) among the chronic pain cohort was 43.9 (±28.0) while the pain-free cohort exhibited a mean FPI score of 20.4 (±20.6) (P<0.0001).

TABLE 11
Cohorts for analytical validation of Clinical Study

| | Healthy | Pain |
|---|---|---|
| Number of subjects | 334 | 153 |
| Male (%) | 50 | 48 |
| Female (%) | 50 | 52 |

| | mean ± SD | | P-value | AUROC | P-value |
|---|---|---|---|---|---|
| Age | 54.6 ± 11.6 | 55.5 ± 11.4 | 0.4273 | | |
| Creatinine | 125 ± 76 | 109 ± 73 | 0.0255 | | |
| Biomarker (μg/mg of creatinine) | | | | | |
| Methylmalonic acid | 1.06 ± 0.99 | 1.49 ± 2.00 | 0.0031 | 0.6191 | <0.0001 |
| Homocysteine | 0.32 ± 0.40 | 0.36 ± 0.29 | 0.3074 | 0.5717 | 0.0124 |
| Xanthurenic acid | 0.46 ± 0.31 | 0.61 ± 0.41 | <0.0001 | 0.6381 | <0.0001 |
| Pyroglutamate | 31.2 ± 12.3 | 44.0 ± 39.1 | <0.0001 | 0.6317 | <0.0001 |
| Vanilmandelate | 3.25 ± 1.29 | 3.42 ± 2.30 | 0.2841 | 0.5560 | 0.0517 |
| 5-HIA | 3.49 ± 3.83 | 3.82 ± 3.55 | 0.3723 | 0.5076 | 0.7930 |
| (5-hydroxyindoleacetatic | | | | | |
| acid) | | | | | |
| Quinolinic acid | 5.38 ± 2.34 | 6.54 ± 3.59 | <0.0001 | 0.5735 | 0.0150 |
| Kynurenic acid | 1.64 ± 0.68 | 2.26 ± 1.13 | <0.0001 | 0.6852 | <0.0001 |
| Hydroxymethylglutarate | 3.12 ± 1.38 | 4.00 ± 2.44 | <0.0001 | 0.6512 | <0.0001 |
| Ethylmalonic acid | 3.02 ± 2.64 | 3.29 ± 3.08 | 0.3266 | 0.5146 | 0.6232 |
| 3-HPMA | 1.44 ± 2.54 | 2.19 ± 3.31 | 0.0061 | 0.5825 | 0.0052 |
| (3-hydroxypropyl | | | | | |
| mercapturic acid) | | | | | |
| FPI Score | 20.4 ± 20.6 | 43.9 ± 28.0 | <0.0001 | 0.7490 | <0.0001 |

Association of FPI scores with clinical assessments of pain: As set forth in Table 12, criterion validation analysis evaluated the significance of any correlations between FPI scores and validated clinical assessments for chronic pain. FPI scores were significantly associated with overall SF-36 scores (P<0.0141), general health (P<0.0457) and even more significantly with emotional wellbeing (P<0.0044), and limitations due to emotional problems (P<0.0011). In the pre-validation model of FPI, scores (0-100 scale) were further categorized into tiers that represent the likelihood of detected abnormalities being pain determinants (Table 13).

TABLE 12
Correlation of FPI score severity and
clinical assessments of chronic pain

| | FPI severity | |
|---|---|---|
| | (Spearman r) | P-value |
| Limitations due to | 0.520 | 0.0011 |
| emotional problems | | |
| Emotional well-being | 0.463 | 0.0044 |
| General health | 0.345 | 0.0457 |
| SF-36 score | 0.406 | 0.0141 |

TABLE 13
Tiers Representing Likelihood Detected Abnormality
Determines Pain for Clinical Study

| | Likelihood of detected | Minimum number of |
|---|---|---|
| | abnormalities being | expected abnormal |
| FPI score | pain determinants | biomarkers |
| 0-19 | LOW | 0 |
| 20-49 | MODERATE | 1 |
| 50-79 | MODERATELY HIGH | 2 |
| 80-100 | HIGH | 3 |

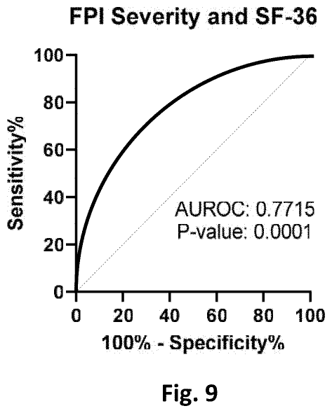

Validation of scoring tiers was carried out by examining associations between SF-36 scores and FPI tiers (low, moderate, moderately high and high). We hypothesized that subjects with severe biochemical disarrangement evident by higher FPI scores would be more impacted, both physically and emotionally by their pain. The SF-36 survey was selected as the most appropriate, validation clinical assessment to evaluate both the physical and mental impact of pain on subjects. FIG. 9 illustrates the ROC curve of the calculated FPI score severity and clinical assessments of chronic pain (i.e., SF-36 scores) in the clinical study. These are the results of validation analysis and highlights the strong association between SF-36 scoring and FPI score as a continuous score (AUROC: 0.7715; P<0.0001). ROC analysis was performed between randomly selected pain patients with moderately-high, high FPI severity scores (>75 FPI; n=20) and low FPI severity scores (<20 FPI; n=20). AUROC: area under the receiver operating characteristic.

TABLE 14
Biomarker and clinical characteristics of subjects across all four FPI Scoring tiers

| | | | MODERATELY | | | |
|---|---|---|---|---|---|---|
| | LOW | MODERATE | HIGH | HIGH | P-value | |
| Minimum | 0 | 1 | 2 | 3 | ANOVA | Linear |
| number of | | | | | | trend |
| expected | | | | | | |
| abnormal | | | | | | |
| biomarkers | | | | | | |
| Average | 0.24 ± 0.44 | 1.84 ± 0.74 | 3.24 ± 0.51 | 4.72 ± 0.46 | <0.0001 | <0.0001 |
| number of | | | | | | |
| abnormal | | | | | | |
| biomarkers | | | | | | |
| SF-36 | 1255 ± 579 | 1662 ± 644 | 1754 ± 555 | 1806 ± 560 | 0.0436 | 0.0177 |
| Scores | | | | | | |

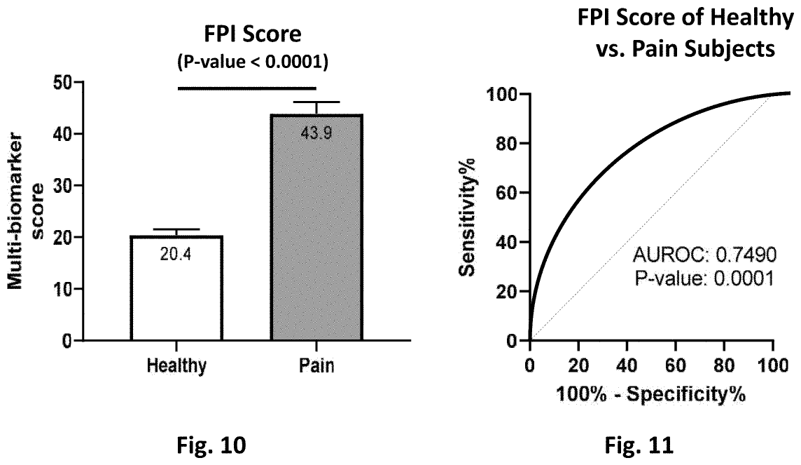

FIGS. 10 and 11 respectively illustrate the comparison of means (non-parametric t-test) of calculated FPI score and ROC curve of the calculated FPI score (healthy vs. pain) observed from the results of this clinical study (i.e., Healthy (n=334) and Pain (n=153) subjects were matched for sex (female: 50%) and age (avg per group: 55 yrs. old)). Multivariate analysis (Least squares) was used to determine distinguishable power of multi-biomarker score (the FPI Score). To achieve normal distributions of biomarkers, box-cox transformations (λ, lambda) and outlier analyses (determined by >2.5*SD) were applied. Subjects were matched for age and sex to control for collection variation between cohorts. Biomarkers are expressed as normalized to creatinine concentrations (m/mg) for each urine sample. Data was analyzed with level of significance at α=0.05. AUROC: area under receiver operating characteristic curve.

Mean comparison testing of FPI scores in pain and healthy cohorts (see FIGS. 10 and 11) also provides evidence of discriminant validity by demonstrating very significant differences in the mean FPI scores from healthy subjects when compared to the chronic pain cohort (P-value <0.0001). Despite strong discriminatory power, this assay is not intended to diagnose chronic pain. Rather, this innovative assay has been designed and validated to identify patients whose pain complaints may be due, at least in part, to biochemical, metabolic and nutritional abnormalities. The importance of identifying such patients with a non-invasive, cost-effective test cannot be overstated as these abnormalities can be treated and corrected with safe, widely available compounds. In addition, patients whose pain is due, at least in part, to underlying biochemical derangement will likely experience no long-term benefit from opioid therapy as this class of medication is in no way addressing the underlying pathology driving the painful symptoms. Only with objective identification of abnormalities and targeted metabolic correction will these patients experience long-term pain relief. Therefore, identification of such patients prior to the initiation of opioid therapy represents an immediately available and cost-effective strategy to reduce the opioid burden.

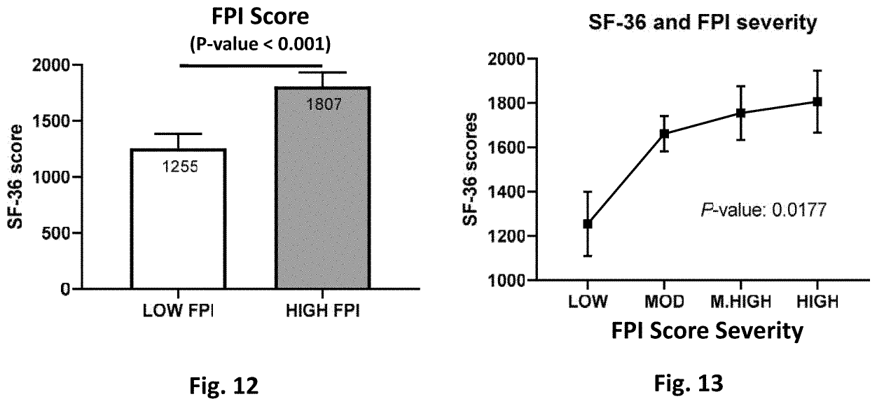

FIG. 12 illustrates the comparison of means (non-parametric t-test) of calculated FPI scores and clinical assessments of chronic pain between pain patients with high FPI severity scores (>75 FPI) and low FPI severity scores (<20 FPI) in this clinical study.

FIG. 13 illustrates the association between SF-36 scores and FPI severity among chronic pain patients in this clinical study. Data is represented as mean±SEM and was analyzed by linear trend analysis of one-way ANOVA. MOD: Moderate; M. HIGH: Moderately High.

The results of the clinical study demonstrate the clinical validity and associated correlations between the FPI scores and validated clinical assessments of chronic pain (limitations due to emotional problems, emotional wellbeing, general health, and overall SF-36 scores) in a heterogeneous cohort of chronic pain patients across multiple clinical sites. The FPI score describes the degree and severity of underlying metabolic derangement that may be driving painful symptomology, and therefore, provides novel objective information that will compliment current subjective assessments. In addition to providing mechanistic insight into underlying, biochemical derangement in chronic pain, the FPI score also directly indicates novel, non-opioid therapies that have been shown to modulate component biomarkers. Novel, biochemical intelligence combined with safe, personalized, non-opioid therapy options will increase the likelihood of successful and prolonged pain management while simultaneously reducing healthcare costs and reliance on opioid medications.

During criterion validation analysis in the clinical study, the FPI score was strongly associated with limitations due to emotional problems (P<0.0011) and emotional wellbeing (P<0.0044), both components of the SF-36 survey. The validity and importance of these findings is further supported by examining the component biomarkers and the diverse set of biochemical and metabolic pathways represented by these surrogate markers. Component biomarkers exhibiting the strongest distinguishing power between healthy and pain cohorts included methylmalonic acid, xanthurenic acid, pyroglutamic acid and the kynurenine pathway metabolites, quinolinic acid and kynurenic acid. While each of these biomarkers lie along pathways that can directly impact the development, worsening and/or perception of pain, they are also intimately linked to mental health and emotional wellbeing.

Considering the high prevalence of mental health disorders including anxiety, depression, bipolar disorder and PTSD in chronic pain populations, it is timely and significant that this clinical study observed and reported prevalent perturbations in biochemical pathways capable of afflicting both physical and mental health. The results provide evidence of criterion validity for the FPI score by demonstrating a significant association with the SF-36 survey and multiple individual components of this validated assessment for chronic pain.

Validity of the FPI score is supported by the direct relationships between component biomarkers and biochemical pathways known to be involved in the pathogenesis of chronic pain. Many of the metabolic, biochemical and nutritional abnormalities indicated by the FPI test have been described in the literature as being underlying causes of pain. As noted above, methylmalonic acid is a sensitive and specific marker of intracellular Vitamin B12 status and elevated levels of this urinary metabolite indicate an increased demand for this critical micronutrient. Vitamin B12 deficiencies are commonly detected in the chronic pain population and can drive painful and neuropsychiatric symptomologies. Xanthurenic acid is a metabolomic biomarker of Vitamin B6 status and elevated levels of this kynurenine pathway metabolite indicate an increased demand for Vitamin B6. Vitamin B6 deficiencies have long been recognized as causes of painful peripheral neuropathies, migraine, chronic pain, depression and other neuropsychiatric diseases. Pyroglutamic acid is a well characterized metabolomic biomarker of glutathione depletion or a reduced glutathione response capacity. Elevated levels of this urinary marker, indicating a need for glutathione support, is a common finding in chronic pain patients taking daily doses of acetaminophen (APAP) as APAP is capable of directly depleting glutathione stores. A reduced glutathione response capacity renders nerve cells susceptible to oxidative damage which can cause or worsen peripheral neuropathies and drive the development of neuropsychiatric disease. Quinolinic and kynurenine acid are neuroactive metabolites of the kynurenine pathway (KP) which can impact the development of pain hypersensitivity and depression through their direct action on NMDA receptors and relationship to serotonin synthesis. The KP is the major pathway responsible for the catabolic degradation of dietary tryptophan. Chronic activation of the KP occurs under conditions of systemic inflammation due to the ability of pro-inflammatory cytokines to directly upregulate this critical pathway. Under normal conditions, a small percentage of dietary tryptophan is utilized for serotonin synthesis but in the presence of a persistent inflammatory response, tryptophan is preferentially shunted down the KP and at the cost of serotonin synthesis. This phenomenon leads to the accumulation of quinolinic acid (leading to NDMA mediated excitotoxicity and depression) and decreased serotonin synthesis which further amplifies heightened pain and depressive symptoms.

The FPI biomarkers represent and evaluate cytokine-mediated chronic inflammation (kynurenine pathway metabolites), oxidative stress (pyroglutamic acid, ethylmalonate, and hydroxymethylglutarate), micronutrient deficiencies (methylmalonic acid, xanthurenic acid, homocysteine), and neurotransmitter turnover (5-hydroxyindoleacetic acid and vanilmandelic acid), all of which have direct links to the development, worsening or heightened perception of pain. In addition, many of the non-opioid, biomarker-modulating compounds that would be directly indicated by the FPI test have exhibited significant pain-relieving effects in numerous randomized, controlled clinical trials. FPI testing will facilitate and simplify patient selection for these important compounds by affording providers the ability to objectively identify patients who exhibit abnormalities and hence require metabolic correction therapies.

The clinical validation study subjects were recruited from diverse geographic locations and exhibited a wide variety of primary pain complaints including, but not limited to, low back pain, lumbar radiculopathy, chronic pain syndrome, and cervicalgia (43%, 31%, 22%, 14% of pain cohort, respectively). In addition to diverse disease characteristics, study subjects were also prescribed a variety of opioid analgesics for pain control. Because of these diverse characteristics, which reflect real-world patient populations, the FPI test can be considered a valid objective assessment for chronic pain patients in the clinic. Considering the complexity and biopsychosocial nature of chronic pain, it is not surprising that composite, multi-biomarker statistical models outperformed individual biomarkers during discriminate validation. Chronic pain subjects exhibited abnormal biochemical function across various relevant pathways indicating systemically perturbed biochemistry. While these abnormalities may be responsible for the onset of pain is some patients, other detected abnormalities may reflect the long-term metabolic cost of chronic pain. Regardless of whether detected abnormalities drove the acute onset of pain or the subsequent chronification, one thing is clear, biologically comprehensive test panels evaluating multiple pathways are better equipped to characterize pain biochemistry than single marker assays.

Strong demonstration of criterion, face and discriminant validity show in this clinical study support the use of this innovative assay as an objective assessment of underlying pain biochemistry to aid with current clinical evaluations. These clinical study findings support the use of FPI scores to identify patients whose pain may be due, at least in part, to underlying biochemical abnormalities related to chronic inflammation, oxidative stress, micronutrient deficiencies and/or abnormal neurotransmitter turnover. Objective identification of patients exhibiting high FPI scores will enable providers to initiate novel conversations and implement innovative treatment plans to correct underlying abnormalities and modulate the course of disease.

Longitudinal monitoring through repeat FPI testing will provide feedback on the efficacy of modulating therapies as well as provide patients with objective updates on their biochemical status over time. Such tools will likely improve compliance and motivate patients to adhere to the metabolic correction protocols. Future studies will seek to determine the impact of FPI testing and subsequent metabolic correction on patients' outcomes in both new and long-term chronic pain patients. Future studies may investigate the impact of FPI testing and metabolic correction following acute injury to determine if the transition from acute to chronic pain can be slowed, or even prevented by optimizing biochemical function to ensure an appropriate inflammatory response.

### Concluding Information for this Patent Disclosure

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of diagnosing and treating distinct biologic components contributing to chronic pain experienced by a patient, the method comprising:
a) obtaining a biologic sample on a sample date from the patient experiencing chronic pain;
b) determining the levels of each of two or more biomarkers found in the biologic sample, wherein the two or more biomarkers are selected from the group: methylmalonic acid, homocysteine, xanthurenic acid, 3-hydroxypropyl mercapturic acid (3-HPMA), pyroglutamate, hydroxymethylglutarate (HMG), quinolinic acid, kynurenine acid, 5-hydroxyindoleacetate (5-HIAA), vanilmandelate (VMA), and ethylmalonic acid;
c) diagnosing the patient as having a nerve health pain component to their chronic pain upon determining an abnormal level of one or all of methylmalonic acid, homocysteine, xanthurenic acid, and 3-hydroxypropyl mercapturic acid (3-HPMA) and then directing the administration of an effective amount of one or more nerve health support compounds to the patient diagnosed as having the nerve health pain component to their chronic pain;
d) diagnosing the patient as having an oxidative stress pain component to their chronic pain upon determining an abnormal level of one or all of pyroglutamate, ethylmalonic acid, and hydroxymethylglutarate (HMG) and then then directing the administration of an effective amount of one or more oxidative stress support compounds to the patient diagnosed as having the oxidative stress pain component to their chronic pain;
e) diagnosing the patient as having a chronic inflammation pain component to their chronic pain upon determining an abnormal level of one or both of quinolinic acid and kynurenine acid and then directing the administration of an effective amount of one or more chronic inflammation support compounds to the patient diagnosed as having the chronic inflammation pain component to their chronic pain; and
f) diagnosing the patient as having a neurotransmitter pain component to their chronic pain upon determining an abnormal level of one or both of 5-hydroxyindoleacetate (5-HIAA) and vanilmandelate (VMA) and then then directing the administration of an effective amount of one or more neurotransmitter support compounds to the patient diagnosed as having the neurotransmitter pain component to their chronic pain.

2. The method of claim 1 wherein the biologic sample is urine, the method further comprising:
a) determining the level of creatinine found in the biologic sample: and
b) normalizing the determined levels of each of the two or more biomarkers found in the biologic sample based on the determined level of creatinine.

3. The method of claim 2 further comprising combining the normalized levels of each of the two or more biomarkers found in the biologic sample into a composite pain index.

4. The method of claim 3 further comprising categorizing the composite chronic pain index into a composite chronic pain range selected from the group comprising: a low likelihood that the patient's biochemistry is contributing to their chronic pain, a moderate likelihood that the patient's biochemistry is contributing to their chronic pain, a moderately high likelihood that the patient's biochemistry is contributing to their chronic pain, and a high likelihood that the patient's biochemistry is contributing to their chronic pain.

5. The method of claim 4 further comprising saving a set of patient pain data in electronic memory in association with the sample date and a unique patient ID, the set of patient pain data including one or more of the composite chronic pain index, the composite chronic pain range, and the normalized levels of each of the two or more biomarkers found in the biologic sample.

6. The method of claim 5 further comprising graphically displaying one or more sets of patient pain data associated with the unique patient ID in order of the sample date associated with each set.

7. The method of claim 3, wherein the composite biomarker score is determined through quantitative assessment of a biological markers of one or more of chronic inflammation, oxidative stress, micronutrient status, neurotransmitter synthesis and turnover.

8. The method of claim 7, wherein the composite biomarker score is determined using a statistical or chemometric technique, preferably multivariate analysis of variance (MANOVA).

9. The method of claim 8, wherein said composite biomarker score is determined by comparing said biomarker levels in pain patients to those of healthy control pain free subjects with no history of chronic pain or opioid use.

10. The method of claim 3, wherein said composite biomarker score is used as a predictive tool to identify patients who have a high probably of responding well to interventional pain therapies such as neuromodulation techniques selected from the group comprising: spinal cord stimulation, intrathecal pump therapy, and peripheral nerve stimulation.

11. The method of claim 3, wherein said composite biomarker score is used to provide a non-opioid pain management plan, comprising the step of administering a treatment that directly modulates and/or targets one or more abnormal biomarker findings.

12. The method of claim 11, wherein said composite biomarker score is provided before, during, or after the step of reducing opioid administration in an individual, preferably wherein said composite marker score is used with an opioid weaning or opioid reduction program.

13. The method of claim 3, wherein said composite biomarker score is used to determine a personalized, non-opioid therapy to increase the efficacy of current pain management techniques, wherein said individual is administered said personalized, non-opioid therapy in conjunction with a traditional pain therapy selected from analgesic medications, intervention pain therapies, physical therapy, NSAID therapy, opioids, or combinations thereof.

14. The method of claim 1, wherein when the patient is determined to have atypical biochemistry that contributes to chronic pain, said individual is administered a composition comprising at least two or, or at least three of, or at least four of, or at least five of, or at least six of, or at least seven of, or at least eight of, or at least nine of, or at least 10 of, or at least 11 of vitamin B3 (preferably as Nicotinamide), preferably in an amount of about 300 mg per serving, vitamin B6 (preferably as pyridoxal-5-phosphate), preferably in an amount of about 5 to about 50 or about 20 mg per serving, vitamin B12 (preferably as methylcobalamin), preferably in an amount of about 250 mcg to about 5000 mcg, or about 1000 mcg per serving, magnesium (preferably as magnesium Glycinate), preferably in an amount of from about 100 to about 1000 mg, or about 250 mg per serving, N-Acetylcysteine, preferably in an amount of from about 50 to about 1000 mg, or about 200mg per serving, ashwagandha root powder, preferably in an amount of about 50 to about 1000 mg, or about 200 mg per serving, curcumin C3 complex, preferably in an amount of from about 50 to about 1000 mg, or about 200 mg per serving, alpha-lipoic acid, preferably in an amount of from about 50 to about 1000 mg, or about 200 mg per serving, coenzyme QlO, preferably in an amount of from about 50 to about 1000 mg, or about 200 mg per serving, acetyl-L-carnitine, preferably in an amount of from 50 to about 1000 mg, or about 300 mg per serving, betaine anhydrous, preferably in an amount of from 50 to about 1000 mg, or about 250 mg per serving.

15. A system for diagnosing and treating distinct biologic components contributing to chronic pain experienced by a patient, the system comprising:
a) Means for obtaining a biologic sample on a sample date from the patient experiencing chronic pain;
b) Means for determining the levels of each of two or more biomarkers found in the biologic sample, wherein the two or more biomarkers are selected from the group: methylmalonic acid, homocysteine, xanthurenic acid, 3-hydroxypropyl mercapturic acid (3-HPMA), pyroglutamate, hydroxymethylglutarate (HMG), quinolinic acid, kynurenine acid, 5-hydroxyindoleacetate (5-HIAA), vanilmandelate (VMA), and ethylmalonic acid;
c) Means for diagnosing the patient as having a nerve health pain component to their chronic pain upon determining an abnormal level of one or all of methylmalonic acid, homocysteine, xanthurenic acid, and 3-hydroxypropyl mercapturic acid (3-HPMA) and then directing the administration of an effective amount of one or more nerve health support compounds to the patient diagnosed as having the nerve health pain component to their chronic pain;
d) Means for diagnosing the patient as having an oxidative stress pain component to their chronic pain upon determining an abnormal level of one or all of pyroglutamate, ethylmalonic acid, and hydroxymethylglutarate (HMG) and then then directing the administration of an effective amount of one or more oxidative stress support compounds to the patient diagnosed as having the oxidative stress pain component to their chronic pain;
e) Means for diagnosing the patient as having a chronic inflammation pain component to their chronic pain upon determining an abnormal level of one or both of quinolinic acid and kynurenine acid and then directing the administration of an effective amount of one or more chronic inflammation support compounds to the patient diagnosed as having the chronic inflammation pain component to their chronic pain; and
f) Means for diagnosing the patient as having a neurotransmitter pain component to their chronic pain upon determining an abnormal level of one or both of 5-hydroxyindoleacetate (5-HIAA) and vanilmandelate (VMA) and then then directing the administration of an effective amount of one or more neurotransmitter support compounds to the patient diagnosed as having the neurotransmitter pain component to their chronic pain.
